SEQUENCE LISTING (69 sequences)

SEQ ID NO: 1 - protein, 22 aa, Artificial Sequence
  heavy chain peptide
GLEWVSYLSSGGGSTYYADSVK

SEQ ID NO: 2 - protein, 22 aa, Artificial Sequence
  daratumumab
GLEWVSAISGSGGGTYYADSVK

SEQ ID NO: 3 - protein, 22 aa, Artificial Sequence
  M-protein
GLEWVSYISSGGGSTYYADSVK

SEQ ID NO: 4 - protein, 11 aa, Artificial Sequence
  patient 1de novo
NSVFLEMNSLR

SEQ ID NO: 5 - protein, 11 aa, Artificial Sequence
  patient 1 germline
NSLYLQMNSLR

SEQ ID NO: 6 - protein, 19 aa, Artificial Sequence
  patient 2 de novo
VSCTASGFSSDTHPMSWVR

SEQ ID NO: 7 - protein, 19 aa, Artificial Sequence
  patient 2 de novo
VSCTASGFSSDTYAMSWVR

SEQ ID NO: 8 - protein, 19 aa, Artificial Sequence
  patient 2 germline
LSCTASGFTFGYYPMSWVR

SEQ ID NO: 9 - protein, 11 aa, Artificial Sequence
  patient 3 de novo
LSCEASGFTFR

SEQ ID NO: 10 - protein, 10 aa, Artificial Sequence
  patient 3 germline
LSCSASGFTF

SEQ ID NO: 11 - protein, 26 aa, Artificial Sequence
  patient 4 de novo
SPPVSVSHVEANSPGQTASLTCSGDK

SEQ ID NO: 12 - protein, 6 aa, Artificial Sequence
  patient 4 germline
PSVSVS

SEQ ID NO: 13 - protein, 13 aa, Artificial Sequence
  Patient 4 germline
PGQTASITCSGDK

SEQ ID NO: 14 - protein, 19 aa, Artificial Sequence
  patient 4 germline
  MISC_FEATURE (7)..(12): any aminoacid
PSVSVSPGQTASITCSGDK

SEQ ID NO: 15 - protein, 16 aa, Artificial Sequence
  patient 5 de novo
MTNMDPVDTATYYCAR

SEQ ID NO: 16 - protein, 18 aa, Artificial Sequence
  patient 5 de novo
LVLTQSPATLSASPNAAK

SEQ ID NO: 17 - protein, 16 aa, Artificial Sequence
  patient 5 germline
MTNMDPVDTATYYCAR

SEQ ID NO: 18 - protein, 14 aa, Artificial Sequence
  patient 5 germline
IVLTQSPATLSLSP

SEQ ID NO: 19 - protein, 17 aa, Artificial Sequence
  patient 5 de novo
LVLTQSAPTLSLSDAPR

SEQ ID NO: 20 - protein, 19 aa, Artificial Sequence
  patient 6 de novo
VELLVESGGDLVQPGGSLR

SEQ ID NO: 21 - protein, 18 aa, Artificial Sequence
  patient 6 de novo
LEVLTQSPGTLSLSPDAR

SEQ ID NO: 22 - protein, 16 aa, Artificial Sequence
  patient 6 germline
LVESGGGLVQPGGSLR

SEQ ID NO: 23 - protein, 16 aa, Artificial Sequence
  patient 6 germline
VLTQSPGTLSLSPGER

SEQ ID NO: 24 - protein, 10 aa, Artificial Sequence
  patient 7 de novo
EDTALFYCVK

SEQ ID NO: 25 - protein, 18 aa, Artificial Sequence
  patient 7 de novo
DLQMTQSPSSLSASVGDK

SEQ ID NO: 26 - protein, 10 aa, Artificial Sequence
  patient 7 germline
EDTAVYYCVK

SEQ ID NO: 27 - protein, 18 aa, Artificial Sequence
  patient 7 germline
DIQMTQSPSSLSASVGDR

SEQ ID NO: 28 - protein, 14 aa, Artificial Sequence
  patient 8 de novo
SMTAADTGVYYCAR

SEQ ID NO: 29 - protein, 18 aa, Artificial Sequence
  patient 8 de novo
QLDLTQSPSSLSASVGDR

SEQ ID NO: 30 - protein, 14 aa, Artificial Sequence
  patient 8 germline
SVTAVDTGVYYCAR

SEQ ID NO: 31 - protein, 17 aa, Artificial Sequence
  patient 8 germline
IQLTQSPSSLSASVGDR

SEQ ID NO: 32 - protein, 16 aa, Artificial Sequence
  patient 9 de novo
LNSAGYLSLQGGVPSK

SEQ ID NO: 33 - protein, 16 aa, Artificial Sequence
  patient 9 de novo
LSLYGASNLQGGVPSK

SEQ ID NO: 34 - protein, 9 aa, Artificial Sequence
  patient 9 germline
SLQSGVPSK

SEQ ID NO: 35 - protein, 16 aa, Artificial Sequence
  patient 9 germline
LLIYSASNLQSGVPSR

SEQ ID NO: 36 - protein, 22 aa, Artificial Sequence
  germline peptide
GLEWVSAISGSGGSTYYADSVK

SEQ ID NO: 37 - protein, 11 aa, Artificial Sequence
  M-prot pept seq
AEDTAVYYCVR

SEQ ID NO: 38 - protein, 11 aa, Artificial Sequence
  de novo m prot seq
EDTAAVYYCVR

SEQ ID NO: 39 - protein, 10 aa, Artificial Sequence
  Germline peptide
EDTAVYYCVR

SEQ ID NO: 40 - protein, 4 aa, Artificial Sequence
  germline peptide 1
EDTA

SEQ ID NO: 41 - protein, 6 aa, Artificial Sequence
  Germline peptide 2
VYYCVR

SEQ ID NO: 42 - protein, 19 aa, Artificial Sequence
  de novo m prot pept
ASGAALLTYLNWYQQTPGK

SEQ ID NO: 43 - protein, 15 aa, Artificial Sequence
  Germline peptide
SISSYLNWYQQKPGK

SEQ ID NO: 44 - protein, 11 aa, Artificial Sequence
  M-prot
  MISC_FEATURE (7)..(7): oxidation
NSVFLEMNSLR

SEQ ID NO: 45 - protein, 19 aa, Artificial Sequence
  M prot
  MISC_FEATURE (3)..(3): carbamidomethylation
VSCTASGFSSDTHPMSWVR

SEQ ID NO: 46 - protein, 19 aa, Artificial Sequence
  M prot
  MISC_FEATURE (3)..(3): carbamidomethylation
VSCTASGFSSDTYAMSWVR

SEQ ID NO: 47 - protein, 11 aa, Artificial Sequence
  M prot
  MISC_FEATURE (3)..(3): carbamidomethylation
LSCEASGFTFR

SEQ ID NO: 48 - protein, 26 aa, Artificial Sequence
  M prot
  MISC_FEATURE (22)..(22): carbamidomethylation
SPPVSVSHVEANSPGQTASLTCSGDK

SEQ ID NO: 49 - protein, 16 aa, Artificial Sequence
  M prot
  MISC_FEATURE (1)..(1): oxidation
  MISC_FEATURE (4)..(4): oxidation
MTNMDPVDTATYYCAR

SEQ ID NO: 50 - protein, 18 aa, Artificial Sequence
  M prot
LVLTQSPATLSASPNAAK

SEQ ID NO: 51 - protein, 17 aa, Artificial Sequence
  M prot
LVLTQSAPTLSLSDAPR

SEQ ID NO: 52 - protein, 19 aa, Artificial Sequence
  M prot
VELLVESGGDLVQPGGSLR

SEQ ID NO: 53 - protein, 18 aa, Artificial Sequence
  M prot
LEVLTQSPGTLSLSPDAR

SEQ ID NO: 54 - protein, 10 aa, Artificial Sequence
  M prot
  MISC_FEATURE (8)..(8): carbamidomethylation
EDTALFYCVK

SEQ ID NO: 55 - protein, 18 aa, Artificial Sequence
  M prot
  MISC_FEATURE (4)..(4): oxidation
DLQMTQSPSSLSASVGDK

SEQ ID NO: 56 - protein, 14 aa, Artificial Sequence
  M prot
  MISC_FEATURE (2)..(2): oxidation
  MISC_FEATURE (12)..(12): carbamidomethylation
SMTAADTGVYYCAR

SEQ ID NO: 57 - protein, 18 aa, Artificial Sequence
  M prot
QLDLTQSPSSLSASVGDR

SEQ ID NO: 58 - protein, 16 aa, Artificial Sequence
  M prot
LNSAGYLSLQGGVPSK

SEQ ID NO: 59 - protein, 16 aa, Artificial Sequence
  M prot
LSLYGASNLQGGVPSK

SEQ ID NO: 60 - protein, 7 aa, Homo sapiens
DTLMISR

SEQ ID NO: 61 - protein, 7 aa, Homo sapiens
YLTWASR

SEQ ID NO: 62 - protein, 8 aa, Homo sapiens
VTSTLTLK

SEQ ID NO: 63 - protein, 16 aa, Homo sapiens
YVTSAPMPEPQAPGRB

SEQ ID NO: 64 - protein, 18 aa, Homo sapiens
TVAAPSVFIFPPSDEQLK

SEQ ID NO: 65 - protein, 15 aa, Homo sapiens
SYSCQVTHEGSTVEK

SEQ ID NO: 66 - protein, 22 aa, Artificial Sequence
  labeled daratumumab
  MISC_FEATURE (22)..(22): stable isotope labeled
GLEWVSAISGSGGGTYYADSVK

SEQ ID NO: 67 - protein, 22 aa, Artificial Sequence
  M-protein labeled
  MISC_FEATURE (22)..(22): stable isotope labeled
GLEWVSYISSGGGSTYYADSVK

SEQ ID NO: 68 - protein, 24 aa, Artificial Sequence
  labeled ipilimumab
  MISC_FEATURE (1)..(1): TMT labeled
  MISC_FEATURE (24)..(24): TMT labeled
TGWLGPFDYWGQGTLVTVSSASTK

SEQ ID NO: 69 - protein, 22 aa, Artificial Sequence
  M-protein labeled
  MISC_FEATURE (1)..(1): TMT labeled
  MISC_FEATURE (22)..(22): TMT labeled
GLEWVSYISSGGGSTYYADSVK

(19) United States
(12) Patent Application Publication
(10) Pub. No.: US 2023/0042129 A1
(43) Pub. Date: Feb. 9, 2023

(54) Method for Monitoring of Deep Remissions in Multiple Myeloma and Other Plasma Cell Dyscrasias

(71) Applicants: Erasmus University Medical Center Rotterdam, Rotterdam (NL); Stichting Radboud Universitair Medisch Centrum, Nijmegen (NL)

(72) Inventors: Joannes Franciscus Maria Jacobs, Nijmegen (NL); Theo Marten Luider, Rotterdam (NL)

(21) Appl. No.: 17/783,861

(22) PCT Filed: Dec. 11, 2020
(86) PCT No.: PCT/NL2020/050776

§ 371 Date: Jun. 9, 2022

(30) Foreign Application Priority Data
Dec. 11, 2019 (EP) 19215329.4

(51) Int. Cl. G01N 33/68; G01N 33/574

(52) U.S. Cl. CPC G01N 33/6854; G01N 33/57426; G01N 33/6848; G01N 2800/52; G01N 2800/54

(57) ABSTRACT

The present invention provides a method for quantifying a monoclonal (M-) protein in a sample of a subject, the method comprising the steps of:—subjecting a serum sample of a subject to serum protein electrophoresis (SPE) in a gel, preferably serum protein electrophoresis in an agarose gel, to separate serum proteins into different serum protein fractions, optionally followed by immunofixation electrophoresis (IFE) and further optionally involving immunostaining of the gel;—excising from said gel a gel part comprising, or suspected of comprising, a M-protein;—performing an enzymatic digestion of proteins present in said gel part in order to provide a peptide digest comprising at least one M-protein peptide;—subjecting said peptide digest comprising said at least one M-protein peptide to liquid chromatography-mass spectrometry (LC-MS) to determine a quantity of said at least one M-protein peptide, thereby quantifying said M-protein in said sample.

### FIELD OF THE INVENTION

The invention is in the field of disease monitoring, in particular in monitoring of progression and remission of plasma cell dyscrasias, such as multiple myeloma. The invention pertains to methods of monitoring M-protein in patient serum samples, in particular by high-resolution mass spectrometry.

### BACKGROUND OF THE INVENTION

M-protein, a monoclonal immunoglobulin (Ig) produced by malignant plasma cells in the bone marrow, is crucial in multiple myeloma (MM) diagnostics and follow-up. In clinical laboratories, M-protein is most commonly detected and quantified with serum protein electrophoresis (SPE), using patient serum, or urine, as the starting material for analysis. In the SPE gel, proteins are separated into five visible fractions: albumin, alpha-1, alpha-2, beta and gamma fraction. In healthy subjects the gamma fraction is broad and diffuse as it contains healthy polyclonal Ig repertoire and the diversity of Ig amino acid sequences. However, in gels from MM samples, a sharp band, called a monoclonal spike (M spike), appears usually in the gamma region of the SPE gel, due to high abundance and monoclonality of the M-protein, above the polyclonal background. Presence of this M-spike indicates presence of the M-protein in the subject sample, and the presence of a (pre)malignant condition or MM.

SPE has a relatively limited sensitivity and cannot detect M-protein at levels lower than 0.5 g/L. With new treatments and treatment strategies increased percentage of MM patients obtain stringent complete remission (CR), defined by non-detectable levels of M-protein by SPE. Although MM remains as an incurable disease, around half of the patients achieve CR. Hence, SPE is not sensitive enough to monitor deep responses in MM. More sensitive techniques to monitor MM are cellular and molecular techniques, such as flow cytometry, next generation sequencing and allele-specific oligonucleotide-qPCR, that are all performed on bone marrow samples.

Preferably, however, sensitive MM monitoring would be performed on blood-based samples, resulting in a minimally invasive assay with a possibility of frequent patient follow-up. Therefore, more sensitive, and blood-based assays are needed to monitor responses in MM that are below the detection limit of conventional M-protein diagnostics.

The present inventors have previously developed a serum-based targeted mass-spectrometry assay to detect M-proteins, even in the presence of three therapeutic monoclonal antibodies. This assay can target proteotypic M-protein peptides as well as unique peptides derived from these therapeutic monoclonal antibodies. It was shown that this mass-spectrometric assay was more than two orders of magnitude more sensitive than conventional, SPE-based, M-protein diagnostics.

However, in targeted mass-spectrometry, it is necessary to select (patient-specific) M-protein peptides as measurement surrogates for the M-protein. To this end, M-protein sequence information is needed, which can currently only be obtained by molecular techniques based on plasma cell assessment, thereby requiring invasive bone marrow aspirations or biopsies. In the context of patient comfort bone marrow biopsies are undesirable, particularly when aiming to monitor disease. Additionally, bone marrow based approaches introduce risk of sampling error due to tumor heterogeneity. Hence there is a need for a method of M-protein diagnostics which is non-invasive, less prone to sampling error and for which no M-protein sequence information is available or required.

Another problem with current M-protein diagnostics is that in order to determine how treatment variations affect disease outcome, prospective cohort studies are needed wherein a group of similar individuals (cohorts) subjected to different treatment variations are followed over time. Such prospective sample cohorts may take years to collect. Hence, there is a need for a method of M-protein diagnostics which can be performed on patient samples that have been collected and archived over a long period of time.

As indicated above, a problem with current M-protein diagnostics is that higher efficacy of new treatment strategies for MM, such as new antibodies and small molecules, demand for more sensitive techniques to detect minimal residual disease (MRD). The importance of MRD for hematologic malignancies, which term indicates a residual presence of a small number of cancer cells in the body after cancer treatment, as a biomarker of tumor burden quantification and as surrogate endpoint marker in regulatory submissions, including in clinical trials, is now broadly recognized. Whereas reduction of tumor burden is a traditional and essential indicator of treatment response, MRD can serve as a surrogate marker and may indicate poor tumor reduction or regrowth of highly proliferative cancer cell clones following treatment. The assessment of MRD as a measure of the depth of the response, now emerges as a potent concept in detecting and defining submicroscopic disease, propelled by advances in cellular and molecular technology platforms and complete responses (CRs) with newer therapies in a larger proportion of patients. As conventional serological techniques have become suboptimal for detecting MRD, there is a need for more sensitive techniques for MRD assessment and standardization of disease-specific biomarker thresholds indicative of MRD, that can be applied in clinical studies and practice. Such a highly sensitive technique for MRD detection can play an important role in clinical trials on therapeutic drugs for a large number of plasma cell dyscrasias.

### SUMMARY OF THE INVENTION

To eliminate the need for bone marrow sampling, the present inventors have developed an MS assay that does not require bone marrow samples, and that can be fully serum-based. For this, the present inventors have developed an MS assay that is based on de novo sequencing to acquire the M-protein sequence information. This method can be applied directly on serum samples. De novo sequencing in proteomics uses information directly from mass spectra in order to identify peptide sequences without using a predefined sequence database. For immunoglobulins, the constant region sequences are known and present in a database, however for the variable regions, no data is known a priori. De novo sequencing makes it possible to, partially, deduce unknown primary structures of proteins, or parts of proteins, not directly inscribed in the genome, such as immunoglobulins and the M-protein. The present inventors thus provide a method for M-protein diagnostics from which bone marrow sampling can be excluded.

To overcome the problem of prospective cohort studies wherein a large number of longitudinal samples must be analysed and made available under comparable analytical conditions, the present inventors have developed an MS-based assay for M-protein diagnostics which is performed on M-protein samples in the form protein electrophoresis gels. The advantage of this method is that it can be performed on stored (dried) SPE gels, which are regularly archived as patient samples. Using M-spike bands from archived SPE gels from clinical laboratories as starting material for the MS analysis, the present inventors have now provided a method capable of retrospectively analyzing treatment efficacy.

The present inventors furthermore provide a method for detecting MRD in subjects suffering from or having suffered from plasma cell dyscrasia, wherein said method is based on M-protein quantification using any of the methods of the invention as described herein.

In a first aspect, the present invention provides a method for quantifying a monoclonal (M-) protein in a sample of a subject, the method comprising the steps of:

subjecting a body sample of a subject to protein electrophoresis in a gel, preferably serum protein electrophoresis (SPE) in an agarose gel, to separate proteins comprised in said body sample into different protein fractions, optionally followed by immunofixation electrophoresis (IFE) and further optionally involving immunostaining of the gel;

excising from said gel a gel part comprising, or suspected of comprising, a M-protein;

performing an enzymatic digestion on proteins present in said gel part or on a protein extract thereof in order to provide a peptide digest comprising at least one M-protein peptide;

subjecting said peptide digest comprising said at least one M-protein peptide to mass spectrometry (MS), preferably liquid chromatography (LC-) MS, to determine a quantity of said at least one M-protein peptide, thereby quantifying said M-protein in said sample by MS.

In a second aspect, the present invention provides a method for quantifying a M-protein in a sample of a subject, the method comprising the steps of:

providing from a gel an excised gel part comprising, or suspected of comprising, a M-protein from a body sample of a subject; wherein said gel is obtained by performing the steps of:
subjecting a body sample of a subject to protein electrophoresis in a gel, preferably serum protein electrophoresis (SPE) in an agarose gel, to separate proteins comprised in said body sample into different protein fractions, optionally including immunofixation electrophoresis (IFE) and further optionally including immunostaining of the gel;

performing an enzymatic digestion on proteins present in said gel part or on a protein extract thereof in order to provide a peptide digest comprising at least one M-protein peptide;

subjecting said peptide digest comprising said at least one M-protein peptide to mass spectrometry (MS), preferably liquid chromatography (LC-) MS, to determine a quantity of said at least one M-protein peptide, thereby quantifying said M-protein in said sample.

In preferred embodiments of the above aspects of the invention, the step of subjecting said peptide digest comprising said at least one M-protein peptide to mass spectrometry (MS) coincides or is preceded by the selection of at least one subject-specific M-protein peptide (i.e. to allow longitudinal monitoring of said subject-specific M-protein peptide between different samples of said subject by MS), and wherein the selection of at least one subject-specific M-protein peptide comprises the steps of:
(i) providing a body sample of said subject comprising a plasma cell producing the M-protein, preferably in the form of a bone marrow sample, determining the amino acid sequence of the M-protein produced by said plasma cell, and selecting a subject-specific M-protein peptide sequence that is predicted to be generated by said enzymatic digestion of said M-protein; or,
(ii) providing the amino acid sequence of the M-protein produced by said subject, and selecting a subject-specific M-protein peptide sequence that is predicted to be generated by said enzymatic digestion of said M-protein; or,
(iii) subjecting said peptide digest comprising said at least one M-protein peptide to MS, determining the amino acid sequence of a multitude of peptides in said peptide digest by de novo peptide sequencing, and selecting a subject-specific M-protein peptide sequence from the sequences determined,
   wherein said method further comprises the step of subjecting said peptide digest comprising said at least one M-protein peptide to targeted MS to thereby quantify the selected subject-specific M-protein peptide in said peptide digest.

In a further preferred embodiment of an aspect of the invention, said method further comprises the steps of

preparing a stable isotope labelled (SIL) variant of said selected subject-specific M-protein peptide as a reference peptide, and

adding said reference peptide to said peptide digest after said step of enzymatic digestion;

wherein the quantity of said at least one M-protein peptide is determined by comparing an MS signal of said selected subject-specific M-protein peptide to an MS signal of said reference peptide, thereby quantifying said M-protein in said sample.

In still a further preferred embodiment of an aspect of the invention, said subject is a patient suffering from, having suffered from, or at risk of suffering from plasma cell dyscrasia, or a patient undergoing treatment for plasma cell dyscrasia.

In yet a further preferred embodiment of an aspect of the invention, said subject is in remission of plasma cell dyscrasia, and suffering from or at risk of suffering from MRD of plasma cell dyscrasia.

In yet another preferred embodiment of an aspect of the invention, said method further comprising the steps of:

providing a second peptide digest from said subject comprising at least one M-protein peptide; wherein the quantity of said at least one M-protein peptide in said second peptide digest is different or the same as the quantity in said (first) peptide digest; wherein said second peptide digest is obtained by performing the steps of:
subjecting a second body sample of said subject to protein electrophoresis in a gel, preferably serum protein electrophoresis (SPE) in an agarose gel, to separate proteins comprised in said body sample into different protein fractions, optionally followed by immunofixation electrophoresis (IFE) and further optionally involving immunostaining of the gel;
excising from said gel a gel part comprising, or suspected of comprising, M-protein from said second body sample;
performing an enzymatic digestion of proteins present in said gel part in order to provide a second peptide digest comprising at least one M-protein peptide;

labeling said first peptide digest with a first tandem mass tag (TMT) label and labelling said second peptide digest with a second TMT label; wherein said first TMT label and second TMT label are of different mass;

mixing said labelled first and labelled second peptide digest; and

subjecting said mixed labelled first and labelled second peptide digest, both comprising said at least one M-protein peptide, to liquid chromatography-mass spectrometry (LC-MS)

quantifying said M-protein in said second body sample as a relative quantity of said M-protein in said first body sample, whereby the M-protein in the first and second sample are quantified by their respective M-protein peptides which are of different mass and have the same LC retention time.

In preferred embodiments of methods of providing first and second M-protein peptide digests from first and second subject body samples, said first body sample of said subject is obtained while said subject is in remission, and said second body sample of said subject is obtained while said subject is in relapse.

In further preferred embodiments of an aspect of the invention, said plasma cell dyscrasia is selected from the group consisting of leukemia or lymphoma of B-cell non-Hodgkin type with plasma cell component; multiple myeloma (MM); plasmacytoma; lymphoplasmacytic lymphoma; AL amyloidosis; monoclonal gammopathy of undetermined significance (MGUS); smoldering multiple myeloma (SMM); macroglobulinemia; Waldenström disease; plasmacytoma; acute lymphoblastic leukemia (ALL); chronic lymphocytic leukemia (CLL); prolymphocytic leukemia (PLL); T-lymphoblastic lymphoma (TLL); acute myeloblastic leukaemia (AML); B-cell lymphoma, and cryoglobulinemia.

In further preferred embodiments of an aspect of the invention, said subject is has been treated with, or is undergoing treatment with, a therapeutic monoclonal antibody or small molecule inhibitor, preferably wherein said therapeutic monoclonal antibody is selected from daratumumab, nivolumab, elotuzumab, denosumab, blinatumomab and/or ipilimumab.

In further preferred embodiments of an aspect of the invention, said MS is high-resolution MS, preferably high-resolution MS performing parallel reaction monitoring (PRM) measurements, more preferably high-resolution MS performing PRM measurements in MS/MS mode, even more preferably high-resolution targeted LC-MS performing PRM measurements in MS/MS mode on an Q-Orbitrap mass spectrometer.

In another preferred embodiment of an aspect of the invention, the enzymatic digestion is performed using a protease, including, but not limited to a serine protease, a cysteine protease, a threonine protease, an aspartic protease, a glutamic protease, a metalloprotease, and an asparagine peptide lyase, more preferably a serine protease. Most preferably the enzymatic digestion is a tryptic or chymotryptic enzymatic digestion using trypsin (EC 3.4.21.4) or chymotrypsin (EC 3.4.21.1).

In another aspect, the present invention provides a method for typing a subject in plasma cell dyscrasia remission as having plasma cell dyscrasia minimal residual disease (MRD) (i.e. is at risk of plasma cell dyscrasia relapse), comprising performing any of the methods according to the present invention as described above on a body sample of said subject, wherein said subject is typed as having plasma cell dyscrasia MRD if M-protein is detected in said body sample using said method.

In another aspect, the present invention provides a method for monitoring a subject having plasma cell dyscrasia MRD, said method comprising performing any of the methods according to the present invention as described above on at least two longitudinal body samples, preferably wherein said at least two longitudinal body samples have been retrieved at different time points that are spaced apart by an interval of at least one or more days, one or more weeks, one or more months, or one or more years.

In another aspect, the present invention provides a standard-of-care therapeutic agent against plasma cell dyscrasia, preferably a therapeutic monoclonal antibody or small molecule inhibitor, more preferably a therapeutic monoclonal antibody selected from daratumumab, nivolumab, elotuzumab, denosumab, blinatumomab and/or ipilimumab, for use in the treatment of a subject suffering from plasma cell dyscrasia MRD, wherein said subject is typed according to a method of the invention or monitored according to a method of the invention.

In another aspect, the present invention provides a method of treating a subject suffering from plasma cell dyscrasia or having plasma cell dyscrasia MRD, comprising the step of:

performing any of the methods according to the present invention as described above in order to determine that M-proteins are present in said subject, and

administering to said subject a therapeutically effective amount of a standard-of-care therapeutic agent against plasma cell dyscrasia; preferably a therapeutic monoclonal antibody or small molecule inhibitor, more preferably a therapeutic monoclonal antibody selected from daratumumab, nivolumab, elotuzumab, denosumab, blinatumomab and/or ipilimumab.

In another aspect, the present invention provides the use of a standard-of-care therapeutic agent against plasma cell dyscrasia, preferably a therapeutic monoclonal antibody or small molecule inhibitor, more preferably a therapeutic monoclonal antibody selected from daratumumab, nivolumab, elotuzumab, denosumab, blinatumomab and/or ipilimumab, in the manufacture of a medicament for the treatment of a subject suffering from multiple myeloma MRD, wherein said subject is typed according to the method of the invention or monitored according to the method of the invention.

In another aspect, the present invention provides the use of mass spectrometry de novo peptide sequencing of M-protein peptides in a peptide digest of M-proteins extracted from a protein electrophoresis gel for identifying subject-specific M-protein peptides in subjects suffering from plasma cell dyscrasia or having plasma cell dyscrasia MRD, preferably by performing the method of any of the methods according to the present invention as described above, in particular using step iii for selection of subject/specific peptides as described.

In another preferred embodiment of an aspect of the invention, the method is a method for typing a subject for multiple myeloma minimal residual disease (MRD); wherein said subject is typed as having multiple myeloma MRD if the Mprotein in said sample is present at detectable levels using the methods of the present invention.

In other preferred embodiments of this aspect, the M-protein is an (intact), preferably monoclonal, antibody, a (free) antibody heavy chain, a (free) antibody light chain, a shortened version of these proteins, or any combination of these proteins. Preferably, increased or elevated levels of M-protein are increased or elevated levels in blood, serum, liquor, or urine.

### DESCRIPTION OF THE DRAWINGS

FIG. 1. Monitoring the M-protein in ten patients, including the reference patient, by serum protein electrophoresis (SPE) and mass spectrometry (MS). SPE results are indicated with the right y-axis and shown in orange; for retrospectively selected patients M-protein concentration equal or less than 5 g/L is plotted at 5 g/L. Targeted MS signals are indicated in arbitrary units on the left y-axis and shown in blue for the heavy chain, and in red for the light chain. Full symbols represent detectable M-protein/M-protein peptide, and empty symbols indicate that the M-protein/M-protein peptide was not detectable, using conventional M-protein diagnostics or MS, respectively. For the reference patient, GLEWVSYLSSGGGSTYYADSVK was plotted for the heavy chain peptide.

FIG. 2. Monitoring peptides from the constant regions of immunoglobulin (Ig) heavy and light chains, for patient 1 (with an IgA lambda M-protein). Peptide abundance from the shotgun mass spectrometry data is plotted on the y-axis.

FIG. 3A-3H. Monitoring peptides from the constant regions of immunoglobulin (Ig) heavy and light chain, for retrospectively selected patients 2-9. Peptide abundance from the shotgun mass spectrometry data is plotted on the y-axis.

FIG. 4. Experimental design for the SPE-MS assay, including serum protein electrophoresis in gel digestion and mass spectrometry measurement. Gel band of interest is cut from the gel and in gel digestion is performed. After peptide extraction, stable isotope labeled (SIL) peptides are added for protein quantification. Samples are cleaned-up with C18 ZipTips and measured with parallel reaction monitoring (PRM) technology.

FIG. 5. Calculation of M-protein concentration from PRM measurements. Concentrations are calculated from the signal ratio between the endogenous M-protein peptide and the stable isotope labelled (SIL) peptide. SIL peptide is an internal standard and known amount is added to the sample; in this experiments 250 fmol of the SIL peptide is added to each sample extracted from the SPE gels. Concentration of M-protein in serum is calculated from 10 μL of serum that are measured in the SPE gel. As IgG is a homodimer, molar mass of an IgG (150 000 g/mol) is divided by two, therefore the molar concentration of the M-protein is multiplied with 75 000 g/mol.

FIG. 6. Experimental design for tandem mass tag (TMT) labeling of peptides extracted from serum protein electrophoretic gel bands. Two samples with high and low M-protein concentration (A) are labeled with two different TMT labels (B), samples are cleaned-up using C18 ZipTips (C), and measured on the mass spectrometer (D). Concentration of the low concentration M-protein sample can now be calculated from the ratio between the two reporter ions and known concentration of the high (initial) concentration serum sample.

FIG. 7. Dilution series of the M-protein in a control serum matrix with spiked daratumumab (1 g/L). Measured concentration of the M-protein in each dilution, corrected for the recovery calculated for the highest concentration M-protein sample (3.3 g/L of the M-protein), is shown on the y-axis. Dilutions were prepared with 5 fold incremental steps and measured in duplicate. Red and green squares represent first and second measurement of daratumumab (GLEWVSAISGSGGGTYYADSVK), respectively. Blue and orange circles represent first and second measurement of M-protein (GLEWVSYISSGGGSTYYADSVK), respectively. Data points for blank measurements (control serum) are plotted at 0.

FIG. 8. Comparison of SPE and IFE as starting materials for mass spectrometry. Two dilutions of daratumumab (0.04 g/L and 1 g/L) in control serum were measured in triplicate on SPE and IFE (IgG lane). Maximum heights of the peaks corresponding to daratumumab endogenous peptide are higher in SPE than IFE (IgG lane) samples for both dilutions. Maximum heights of the peaks for the stable isotope labeled (SIL) peptide, which was added in the same concentration in all samples, were measured as a control and they do not show this difference in maximum peak height.

FIG. 9. Tandem mass tag (TMT) labeling results for the mixture of two samples with different M-protein concentrations (A), with added 1 g/L of ipilimumab (B). High concentration M-protein sample (3.3 g/L of M-protein) was labeled with TMT6-127 and low concentration M-protein sample (5.3 mg/L of M-protein) was labeled with TMT6-126. Both reporter ions are detected in the MS2 spectra.

### DETAILED DESCRIPTION OF THE INVENTION

With new treatment strategies and more patients reaching stringent complete remission in multiple myeloma (MM), there is an increasing need for sensitive residual disease monitoring. A sensitive, blood-based assay allows frequent MM monitoring and aids in early recognition of disease progression. Mass spectrometry (MS) is a known method to measure M-protein in blood-based samples from MM patients. The presently proposed method, however, is capable of detecting patient-specific M-protein at such low levels that deep response monitoring is now feasible hundreds of days earlier than conventional methodologies. This means that relapse is detected much earlier allowing immediate therapeutic intervention.

In order to monitor M-protein levels by the use of MS, it is required that the M-protein sequence is known in order to select patient-specific M-protein peptides that will be used as surrogates for measuring the M-protein in the patient sample. Such sequences are not always available. In one preferred embodiment of the present invention, de novo proteomic sequencing may be performed, using the excised M-protein band from diagnostic protein electrophoretic gels as the starting material for the analysis, to acquire M-protein sequence information from (individual) patients.

In all patients studied in the Examples described herein below, at least one patient-specific M-protein peptide was identified using de novo sequencing in combination with high-resolution MS. By performing de novo sequencing, instead of DNA/RNA sequencing, the need for bone marrow sampling is eliminated and the assay for M-protein monitoring as proposed herein in a preferred embodiment is therefore entirely blood-based. Additionally, superior sensitivity of the de novo sequencing MS assay, compared to serum protein electrophoresis, enables an average of 340 days earlier detection of disease progression.

In the section below, a detailed description of the method of the invention will be provided. In certain instances, the description is exemplified by detection and (qualitative or quantitative) quantification of M-protein in multiple myeloma. One of skill will understand that aspects of the invention are equally applicable to other plasma cell dyscrasias.

Unless otherwise noted, the terms used herein are to be understood according to conventional usage by those of ordinary skill in the relevant art. In addition to the definitions of terms provided below, definitions of common terms in molecular biology may also be found in: Rieger et al, (1991) Glossary of genetics: classical and molecular, 5th Ed., Berlin: Springer-Verlag; and in Current Protocols in Molecular Biology; F. M. Ausubel et al, Eds., Current Protocols, a joint venture between Greene Publishing Associates, Inc. and John Wiley & Sons, Inc., (1998 Supplement).

The term “sample”, as used herein, refers to any sample from a subject, including but not limited to blood samples, plasma and serum samples, urine samples, bone marrow samples, spinal fluid or liquor samples, or sections of tissues, e.g. obtained by surgery or biopsy. In preferred embodiments of aspects of the present invention, the sample of a subject is a blood or serum sample. The skilled person is well aware of suitable methods to retrieve a blood sample from a subject, for example via venipuncture.

The term “subject”, as used herein, refers to a mammal, preferably a primate, more preferably a human. In the context of the invention, the term “subject” preferably refers to a human, suffering or suspected to suffer from a plasma cell dyscrasia as defined herein, or a human, suffering or suspected to suffer from MRD associated with said plasma cell dyscrasia.

The term “paraprotein”, also referred to herein as M-protein, monoclonal protein, or myeloma protein, which terms are interchangeable, refers to the immunoglobulin product of a single plasma cell clone (monoclonal paraprotein) in plasma cell dyscrasias. In healthy subjects, all plasma cells produce immunoglobulins (also called gammaglobulins), which consist of a heavy chain (IgG, IgA, IgM, IgD or IgE) and a light chain (kappa or lambda) linked together. One plasma cell produces one type of immunoglobulin (for instance, IgA kappa or IgG kappa). In the case of plasma cell dyscrasia, the monoclonal malignant plasma cells all produce the same immunoglobulin. This monoclonal immunoglobulin is referred to as M-protein or paraprotein which usually consists of a heavy chain (most often IgG, but also IgA or IgM, and very rarely IgD and IgE, also referred to as y, α, μ, δ, and ε type heavy chain, respectively) and a light chain (kappa [κ] or lambda [λ]). As the paraprotein is composed of immunoglobulins all having the same size and charge characteristics, it migrates as a discrete band on serum or urine electrophoresis. Paraproteins can be found in body samples (e.g. blood or urine) of almost all patients with plasma cell dyscrasia and subjects suffering from MRD of plasma cell dyscrasia. The M-protein is one of the oldest and best tumor markers of MM. The amount of paraprotein in a body sample may be used to measure the tumorload.

In some forms of MM, the malignant cells may no longer produce complete immunoglobulins, but only produce (kappa or lambda) free light chains not bound to a heavy chain. These free light chains can be detected in serum, but are mostly excreted in the urine as they are not retained by the glomerular basement membrane. Light chains in urine are also referred to as urine M-protein or Bence-Jones. In light chain disease the amount of M-protein in urine is used to measure the tumorload. The serum free light chain assay, has recently been recommended by the International Myeloma Working Group for the screening, diagnosis, prognosis, and monitoring of plasma cell dyscrasias.

The paraprotein in aspects of this invention may thus be an intact immunoglobulin (antibody), an immunoglobulin light chain, or an immunoglobulin heavy chain, or a part of the above.

In some (rare) forms of MM, referred to as non-secreting myeloma, the malignant plasma cells do not produce quantifiable amounts of monoclonal immunoglobulin or free light chains. Consequently the tumorload can not be measured by the amount of M-protein in serum or urine, but must instead be measured by the amount of plasma cells in bone marrow smears or bone marrow biopsy. Methods targeted at diagnosing non-secreting myeloma are not an aspect of this invention.

The term “paraproteinemia” as used herein, refers to the phenomenon of increased, elevated or excessive levels of an immunoglobulin (paraprotein or a part thereof) in a body sample, usually blood or urine, relative to reference levels in healthy subjects. The term “paraproteinemia” as used herein may be used interchangeable with the term “gammopathy”. The gammopathy may be a biclonal or monoclonal gammopathy, preferably a monoclonal gammopathy. The term does preferably not include reference to polyclonal gammopathy. The disease condition (dyscrasia) underlying the paraproteinemia is usually a (blood) plasma cell dyscrasia, mostly due to an immunoproliferative disorder or hematologic neoplasm, including leukemia and lymphoma of various type, but usually B-cell non-Hodgkin lymphoma with a plasma cell component, including multiple myeloma, plasmacytoma, and lymphoplasmacytic lymphoma; and amyloid light-chain (AL) amyloidosis (also known as primary systemic amyloidosis (PSA) or primary amyloidosis). The plasma cell dyscrasia may include a spectrum of progressively more severe monoclonal gammopathies in which a clone or multiple clones of pre-malignant or malignant plasma cells (sometimes in association with lymphoplasmacytoid cells or B lymphocytes) over-produce and secrete into the blood stream a paraprotein, i.e. an abnormal monoclonal antibody or portion thereof.

The term “plasma cell dyscrasia”, as used herein, thus refers to any disease underlying the paraproteinemia, including but not limited to leukemias and lymphomas of various types, in particular B-cell non-Hodgkin lymphomas with a plasma cell component; multiple myeloma (MM); plasmacytoma; lymphoplasmacytic lymphoma; AL amyloidosis; monoclonal gammopathy of undetermined significance (MGUS); smoldering multiple myeloma (SMM); macroglobulinemia; Waldenström disease; plasmacytoma; acute lymphoblastic leukemia (ALL); chronic lymphocytic leukemia (CLL); prolymphocytic leukemia (PLL); T-lymphoblastic lymphoma (TLL); acute myeloblastic leukaemia (AML); B-cell lymphoma, and cryoglobulinemia. A plasma cell dyscrasia in aspects of this invention is preferably a plasma cell dyscrasia associated with paraproteinemia.

The term “multiple myeloma”, abbreviated MM, as used herein, and also referred to as active MM or Kahler's disease, is a hematological cancer characterized by the accumulation of neoplastic plasma cells in the bone marrow associated with elevated serum and/or urine monoclonal paraprotein (M-protein) levels. Clinical manifestations of MM include lytic bone lesions, anemia, immunodeficiency, hypercalcemia, and renal function impairment. MM accounts for more than 10% of all hematological malignancies, representing the second most frequent blood cancer in the United States. Neoplastic plasma cells cause elevated levels of monoclonal proteins of varying types in the blood, urine, and organs, including but not limited to M-protein and other immunoglobulins (antibodies), and free light chains, resulting in abnormally high levels of these albumin, and beta-2-microglobulin.

MM is usually preceded by pre-malignant stages that are free of clinical manifestations, referred to as monoclonal gammopathy of undetermined significance (MGUS) and smoldering multiple myeloma (SMM). These pre-malignant disorders are characterized by monoclonal plasma cell proliferation in the bone marrow, with associated M-protein levels in serum and urine, in the absence of end-organ damage or clinical manifestations (asymptomatic).

MGUS, defined by the presence in the serum of M-protein below 30 g/L, an accumulation of less than 10% plasma cells in the bone marrow, in the absence of, almost always precedes MM. MGUS is present in 1% of adults over age of 25 and evolves toward malignant MM at a rate of 0.5-3% per year.

Smouldering myeloma is characterised by serum M-protein levels above 30 g/l or urinary monoclonal protein above 500 mg per 24 h, and/or clonal plasma cells between 10% and 60% on bone marrow biopsy, and no evidence of end organ damage that can be attributed to plasma cell disorder and no myeloma-defining event (above 60% plasma cells in bone marrow or Involved/Uninvolved light chain ratio>100).

In a small percentage of multiple myeloma cases, further genetic and epigenetic changes lead to the development of a plasma cell clone that moves from the bone marrow into the circulatory system, invades distant tissues, and thereby causes the most malignant of all plasma cell dyscrasias, plasma cell leukemia.

The term “typing”, as used herein, refers to determining the level of a biomarker as a qualitative measure of the presence or absence of disease, or as a quantitative measure on the severity of disease or level of remission. In one preferred embodiment of aspects of the invention, typing may occur by determining the presence or absence of a paraprotein in a sample. If said paraprotein is present, said subject is typed as suffering from paraproteinemia, indicative of a plasma cell dyscrasia, as defined herein. In alternative preferred embodiments, typing occurs by determining the relative or absolute amount of paraprotein in a sample. Measurement of a relative amount may be used to detect increased or decreased amounts of paraprotein relative to an earlier sample of the same subject, a reference sample of a subject suffering from a plasma cell dyscrasia, or a reference sample of a healthy individual. An increased amount may indicate of progression or relapse, wherein the degree of elevation may reflect disease activity, while a decreased amount may indicate remission of the plasma cell dyscrasia. Measurement of an absolute amount may also be indicative of the severity of the disease and may allow prognosis of the plasma cell dyscrasia. Any of the above typing methods may be used to detect deep remissions, and may be used to detect MRD. Typing of paraproteinemia using the highly sensitive technique of the present invention may redefine MRD in plasma cell dyscrasias, as it sets new low levels of detection or paraproteins.

The term “minimal residual disease”, abbreviated “MRD”, as used herein, refers to small numbers of leukaemic cells (cancer cells from the bone marrow) causing the plasma cell dyscrasia that remain in the subject during treatment, or after treatment when the patient is in remission (i.e. when no symptoms or signs of disease are present). MRD is the major cause of relapse in plasma cell dyscrasia. In aspects of this invention, MRD is indicated by the minimal residual presence of the paraprotein biomarker associated with said plasma cell dyscrasia. When using the methods of the present invention, MRD associated with plasma cell dyscrasia may be detected at levels 100, 200, 300, 400, 500, 600, 700, 800, 900, 1000, or more, times more sensitive than those of standard SPE procedures for paraprotein detection as described above. Currently, that detection level is considered to be around 0.10 and 0.40 g/L for SPE. The detection limit for MRD associated with plasma cell dyscrasia using the methods of this invention are as low as 0.1-1 mg/L.

The term “detecting”, as used herein, refers to both the qualitative and quantitative measurements of a paraprotein biomarker or plasma cell dyscrasia in the broadest sense.

Typing, quantifying, screening, monitoring and/or diagnosis of monoclonal proteins envisioned in aspects of this invention may be applied for any plasma cell dyscrasia, including MM, MGUS, and SMM, preferably MM, and are preferably used to investigate or detect early relapse or to investigate efficacy of intervention strategies. Further, methods envisioned in aspects of this invention may be used to type, quantify, screen, monitor and/or diagnose a variety of other monoclonal gammopathies including, for instance Waldenström disease and AL amyloidosis.

Treatment of monoclonal gammopathies, in particular multiple myeloma, is focused on therapies that decrease the clonal plasma cell population and consequently decrease the signs and symptoms of disease. If the disease is completely asymptomatic (i.e. there is a paraprotein and an abnormal bone marrow population but no end-organ damage), as in smouldering myeloma, treatment is typically deferred, or restricted to clinical trials. Bone marrow stromal cells are well known to support multiple myeloma disease progression and resistance to chemotherapy. Disrupting the interactions between multiple myeloma cells and stromal cells is an additional target of multiple myeloma chemotherapy. Treatment may either involve direct targeting of MM clones or may be aimed at providing a positive anti-tumor effect by targeting the MM micro-environment. Any biologic may be used for that purpose, include the administration of daratumumab, nivolumab and/or ipilimumab, with or without dexamethasone.

The term “standard-of-care therapeutic agent against plasma cell dyscrasia”, as used herein, refers to any (bio)pharmaceutical therapeutic agent or treatment modality currently used or proposed for potential future use in the treatment of a plasma cell dyscrasia. The term is therefore equivalent to a “medicament for treating plasma cell dyscrasia”. Suitable standard-of-care therapeutic agents against plasma cell dyscrasia may be selected from:
chemotherapeutic drugs, including but not limited to vincristine, cyclophosphamide, cytarabine, bendamustine, daunorubicin, (lipisomal) doxorubicin, idarubicin, mitoxantrone, pirarubicin, granulocyte stimulating factor (CAG), etoposide, melphalan, cytarabine, L-asparaginase, 6-mercaptopurine, methotrexate, methoterate, and teniposide;
monoclonal antibodies, including but not limited to daratumumab, nivolumab, denosumab, elotuzumab, ipilimumab, and blinatumomab;
small molecule inhibitors, including but not limited to dasatinib, imatinib, gefitinib, erlotinib, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, nvp-auy922, obatoclax, navitoclax, and ixazomib;
immunomodulators, including but not limited to thalidomide, lenalidomide, and pomalidomide;
histone deacetylase (HDAC) inhibitors, including panobinostat, vorinostat, romidepsin and belinostat;
radiation therapy;
stem cell transplantation and bone marrow transplant;
any of the above with or without corticosteroids including but not limited to dexamethasone and prednisone;
any combination of the above.

The present inventors have shown that the combination of gel electrophoretic separation of M-protein from other immunoglobulins or free light chains in a sample will lower the detection limit considerably. In fact, as will be shown in the Experimental part below, a much earlier discovery of the presence of minimal residual disease (MRD), potentially resulting in relapse, and concomitant therapeutic control of the disease, is now possible. The invention allows for an improvement of the detection limit by a factor of 1000. Further purification of the immunoglobulin (IgG, IgA or IgB) fraction prior to MS analysis allows for a further improvement of the sensitivity by a factor of 10. Such purification may comprise the use of suitable systems for purification of antibodies from serum and other sample types. One suitable system is Melon Gel resin (Pierce, Rockford, Ill.).

In methods of the present invention, MS-based detection of M-protein is combined with electrophoretic separation of proteins from a subject's body sample, preferably serum or urine. In methods of the invention, gel electrophoresis is preferably performed using agarose as the electrophoresis matrix for protein separation (e.g. 0.5-2 wt % agarose, preferably 0.8-0.9 wt. % agarose, preferably at pH 9-9.2). It is preferred that such methods are highly standardized, as this facilitates longitudinal sample comparison between stored (e.g. archived) samples. The samples are preferably stored in the form of dried electrophoresis gels. A very suitable system for gel electrophoretic separation of proteins is the commercially available Sebia Hydragel® system (Sebia UK Ltd., Camberley, United Kingdom), which is a certified diagnostic system for M-protein diagnostics, and which and can be used in accordance with the manufacturers instructions. The system has a detection limit for M-protein of about 0.1-3 g/L.

Protein electrophoresis is routinely used in clinical laboratories for screening samples for protein abnormalities (e.g. gammopathies). In methods of the present invention, wherein MS-based detection of M-protein is combined with electrophoretic separation of proteins, the proteins may be separated by charge and/or size by applying an electric field across the gel to move the charged molecules through the gel matrix. In this way, most proteins in a body sample, e.g. urine or serum) are separated into 6 major fractions: albumin, alpha-1, alpha-2, beta-1, beta-2, and gamma. The M-protein may be recognized as an M-spike in any of the alpha, beta, or gamma fractions, i.e. as a monoclonal band migrating in any of these electrophoresis zones. Most commonly, the M protein is in the gamma fraction.

The procedure of protein electrophoresis prior to MS analysis may in general comprise one or more of the steps of (not in limiting order): (i) sample preparation and application to the electrophoresis gel, (ii) electrophoretic migration, (iii) drying of the gel, (iv) staining of the separated proteins (e.g. with amidoblack dye), (v) destaining, and optionally (vi) final drying, and optionally (vii) storage (preferably under dry conditions).

Drying and optional final drying of electrophoresis gels facilitates their archiving. Conditions for drying and optional final drying of the spend electrophoresis gels (i.e. after separation of the proteins therein) are not critical. In preferred embodiments of the invention, agarose is used as the electrophoresis gel matrix. Drying of agarose gels may for instance occur at elevated temperatures, for instance at between 50-80° C., for a duration of between 4-30 minutes, preferably at about 65° C., for about 10 minutes. As indicated above, it is preferred that methods are highly standardized, as this facilitates longitudinal sample comparison between stored (e.g. archived) samples. Standardized gel electrophoresis systems are preferably used, such as the Sebia Hydragel® system, and the use of semi-automated procedures for performing the analytical steps of sample application, electrophoresis, drying, staining, destaining and final drying is highly preferred. Such systems are commercially available, and include for instance the Hydrasys device (Sebia, Evry, France), wherein drying and final drying is performed in the device according to manufacturer's protocols. Dried gels may be stored in an archive, preferably under dry conditions, preferably at room temperature.

In practice, a body sample (e.g. blood can be collected in a tube with a clot activator, and after separation from blood components, the serum can be provided as body sample) is loaded on an electrophoresis gel (e.g. in the case of serum, a paper carrier treated with agarose gel) and an electric field is placed across the gel to force migration of proteins across the matrix in the presence of a buffer solution (i.e. in an electrophoretic cell). The various proteins are then separated based on charge. After completion of separation, the gel is dried, fixed to prevent further diffusion of proteins, stained to visualize various protein bands (Coomassie brilliant blue is a common staining agent to visualize bands in serum protein electrophoresis), and then dried for storage. The staining may further comprise immunofixation. These methods are well known to one of skill and are for instance described in Dasgupta, A.; Wahed, A.: Chapter 22—Protein Electrophoresis and Immunofixation. In Clinical Chemistry, Immunology and Laboratory Quality Control; Dasgupta, A., Wahed, A., Eds.; Elsevier: San Diego, 2014; pp 391-406, which is incorporated herein by reference in its entirety.

For subsequent MS analysis, following completion of electrophoresis and optional storage, the gel region of interest comprising the M-protein band is physically cut from the gel. For digestion of the M-protein comprised in the excised gel fragments, the gel fragment is washed and dried, and following a treatment with a cleavable detergent (e.g. RapiGest SF^{(R)}), subjected to in-gel digestion (e.g. with trypsin). The peptide digest is thereafter extracted from the gel matrix and optionally further desalted, concentrated and/or purified (e.g. using C18 chromatographic binding and elution).

In aspects of the present invention, one may perform protein electrophoresis, or one may suitably use an archive of electrophoresis gels provided by other laboratories for subsequent MS analysis of the M-protein band present therein. Aspects of this invention then comprise the step of providing, or having provided, an archive comprising previously prepared electrophoresis gels containing the M-protein in one of the Alpha, Beta, or Gamma fractions. One may then excise gel regions comprising the M-protein band from such archived gels for subsequent further analysis. Alternatively, one may use an archive of previously excised gel regions containing the M-protein provided by other laboratories for subsequent MS analysis of the M-protein band present therein. Aspects of this invention then comprise the step of providing, or having provided, an archive comprising previously excised gel regions containing the M-protein.

Sample material used in aspects of this invention include archival diagnostic SPE gels from MM patients. For such SPE analysis, MM patient serum may have been processed and used for diagnostic purposes. The archived SPE gels may have been stored as dried agarose gels.

The patients whose diagnostic SPE gels are used may be retrospectively selected based on the data in the laboratory information system. Suitable criteria for selection may include SPE detectable M-protein levels (>1 g/L by densitometry), presence of at least one period of remission (characterized by the absence of M-protein by SPE/IFE), and/or presence of at least one episode of relapse with detectable M-protein (>1 g/L by densitometry). It is not necessary that M-protein DNA/RNA sequence data from these patients is available.

Excision and sample preparation of M-protein bands generally includes cutting the M-protein bands from the electrophoresis gels. In cases where the (retrospectively selected) patients is in remission, excision must occur based on a patient-specific cutting template to ensure that the correct area of the gel (i.e. the correct Ig fraction) is cut. In some instances, the M-protein band may not be visible by normal staining of the electrophoresis gel. For this purpose, one may, for instance, use the distance between the albumin band and a detectable M-protein band optionally in combination with the width of the M-protein band from a “positive” electrophoresis gel, as markers for those instances that M-protein is not detectable by gel electrophoresis.

Excised bands may be washed (e.g. with water, 50% acetonitrile (ACN), 100% ACN, and 50% ACN/100 mM ammonium bicarbonate), dried (e.g. in SpeedVac), reduced and alkylated (e.g. by treating with dithiothreitol, followed by treating with iodoacetamide), and again washed and dried as described above.

Subsequently, the washed and dried gel excisions may be subjected to tryptic digestion (preferably overnight). Tryptic peptides may then be extracted (e.g. with trifluoroacetic acid (TFA)/ACN) and dried. The resulting tryptic peptides mix may then be resuspended (e.g. in TFA), and residual gel material may optionally be removed.

The tryptic peptides mix comprising M-protein peptides may then be subjected to liquid chromatography (LC)-mass spectrometry (MS). LC may suitably comprise use of a C18 column. Following the LC step, the sample is subjected to shotgun MS measurements using high resolution Mass Spectrometry, such as e.g. an Orbitrap (Thermo Fisher Scientific, Bremen, Germany), to obtain high resolution full scan MS data, preferably in a mass range of 375.00-1500.00 m/z. MS/MS may then be performed on selected (e.g. the top 20 most intense) monoisotopic masses in the full scan spectra.

Parallel reaction monitoring measurements may then be performed. Targeted MS/MS spectra may be obtained by HCD fragmentation during which MS/MS spectra are collected in centroid mode. All targeted measurements were unscheduled. m/z, charge states, fragment types of selected peptides are listed in Supplementary Information in Table S-1.

De novo sequencing may be performed on shotgun and targeted MS raw data files using commercially available dedicated software, for instance Peaks Studio 6.0 (Bioinformatics Solutions Inc., Waterloo, Canada), in order to acquire M-protein peptide sequences. In addition, a commercial search tool (e.g. Peaks DB database search tool) may be used for constant Ig peptide identification.

In order to select patient-specific M-protein peptides from de novo sequencing data the following procedure may be used. First time points in the disease course, of each patient, may be measured in order to select patient-specific M-protein peptides with the most correct de novo sequence. Peptide candidates have to fulfill two criteria. First, patient-specific M-protein peptide candidate has to be high abundant in a certain patient and absent or low abundant in samples of other patient. Progenesis QI (Waters, Milford, Mass.) may be used for label-free quantitation of MS data. Shotgun MS raw data of the first time point of each patient may be imported into Progenesis with selection criteria for peptide ions: charge between 2+ and 4+, and at least two isotopes. Peptide raw abundances may be exported for further filtering, for which Microsoft Excel 2010 (Microsoft, Redmond, Wash.) may be used. Criteria for peptide filtering: peptide is high abundant for a certain patient and absent or low abundant (<1%) in other patients, peptide has an identification score above 7.

Second, sequence of the peptide candidate has to resemble an Ig germline sequence. Searching for Ig-looking peptides may be performed using dedicated software known to one of skill (e.g. the IgBlast tool; www.ncbi.nlm.nih.gov/igblast/). If no resemblance to Ig germline sequences is found for a certain de novo selected peptide sequence (e.g. as indicated by the IgBlast tool), that peptide may not be selected as an M-protein peptide of interest. Selected M-protein peptide of interest sequences preferably have a sequence similarity percentages above 60%, preferably above 65%, 68%, 70% or 75%.

Longitudinal monitoring of the M-protein and immunoglobulins is now possible at very low M-protein levels. It is shown in the Examples that patient-specific M-protein peptides, acquired from de novo sequencing of the shotgun MS data, can be used to monitor M-protein during the disease course of each patient. The samples obtained during or available for the disease course can suitably be measured with targeted MS, for instance as described in the Examples, using a selected patient-specific M-protein peptide. Targeted MS data can for instance be analyzed using dedicated software (e.g. in Skyline). In order to follow a disease course, the maximum height of the peaks corresponding to the patient-specific M-protein peptides can be plotted for every time point.

Monitoring of Igs during the disease courses of MM patients may be performed on peptides from the constant regions of heavy (IgG, IgA, IgM) and light (kappa and lambda) Ig chains. Suitable peptides may be identified using the a database search (e.g. as present in the Peaks DB program).

In the experimental section below, it is shown that the present invention provides a method for monitoring of deep remissions in multiple myeloma. These examples are only meant to illustrate the applicability of the invention. One of skill in the art will recognize that variations and even deviations from the preferred embodiments of the invention is possible without deviating from the scope of the present invention, and that separate embodiments can be combined.

In aspects of this invention, patient-specific M-protein peptides may be monitored using MS on the M-spike band of PE gels, without using bone marrow samples, which makes the method of the invention, at least in its potential, completely serum-based. Bone marrow sampling is invasive and potentially non-representative, e.g. because of patchy occurrence of tumor cells in the bone marrow. Serum-based assays for monitoring M-protein are preferred as they allow for more frequent sampling and have better patient acceptance, due to their less invasive nature.

In several embodiments of this invention, one or more of the peptides in the M-protein digest analysed by targeted MS analysis, meaning that only pre-selected, specific peptides are analysed (e.g., having mass spectra and retention times). This allows for increased selectivity and sensitivity of the method by limiting the amount of data measured. Other peptides present in the samples will not interfere with the analysis. This is of benefit when samples of patients undergoing treatment with therapeutic antibodies are analysed. The pre-selected, specific peptides can be quantified against a background of highy related peptides of other (therapeutic) immunoglobulins A typical mass spectometric targeted analysis method may be based on selected reaction monitoring (SRM) on a triple quadrupole instrument. In preferred embodiments parallel reaction monitoring (PRM) measurements are performed. PRM is preferably based on Q-Orbitrap as the representative quadrupole-high resolution mass spectrum platform.

The peptides selected for MS targeted analysis and used as surrogates for M-protein measurement may be selected based on available (database) sequence data or MS de novo sequencing. The sequence of a peptide selected for MS targeted analysis based on MS de novo sequencing preferably has a 50%, more preferably 60%, 70%, 80%, 90% or higher sequence similarity to any known immunoglobulin sequence over the length of the peptide, so as to ensure that the peptide is M-protein derived. The sequence of a peptide selected for MS targeted analysis is a peptide sequence that is the product of a digestion with the selected protease. For instance, if using trypsin, the selected peptide sequencehas to be tryptic, i.e. ending in amino acids arginine (R) or lysine (K). Trypsin is the most commonly used enzyme for protein digestion. Furthermore the peptide sequence of the selected peptide is preferably between 10-30 amino acids in length, more preferably between 11-26 amino acids in length. It may me appreciated that shorter peptides (<20 amino acids) are more likely to provide optimal sensitivity. In addition, the peptide sequence of the selected peptide is preferably unique for the protein of interest and thus, in the case of M-proteins which sequences are clone-specific, also patient-specific, making the MS assay a personalized assay for MM monitoring. To select peptides for M-protein monitoring, that satisfy all mentioned criteria, the M-protein sequence has to be available and it is usually acquired from DNA/RNA sequencing from bone marrow samples.

To eliminate the need for bone marrow sampling, the present inventors propose the use of de novo proteomic sequencing. De novo sequencing, from MS data acquired from PE gel samples, is capable of yielding peptides that are suitable for monitoring all individual patients in the study shown (Example 1). Additional validation was obtained by including a reference patient to the patient cohort. For the reference patient, the DNA sequence of the heavy chain was obtained from a bone marrow sample and extensive characterization by proteomics was performed as described earlier (Zajec et al, J Proteome Res. 2018; 17(3):1326-33). Essentially, the same patient-specific M-protein peptide was selected from just de novo sequencing data, as when DNA sequencing was used previously (GLEWVSYISSGGGSTYYADSVK), which shows the satisfactory outcome of de novo sequencing. It is known that de novo sequencing is less accurate than DNA sequencing. However, it is surprisingly shown that the de novo sequence are acceptable in the proposed methods of this invention as they do not interfere with the monitoring of M-protein peptides over time. The mass spectrometer is configured to isolate peptides of a given mass and monitor the signal from specific fragment masses. The instrument measures these signals irrespective of the underlying sequence, and they are meaningful for the peptide concentration. The relevant masses are established based on a diagnostic sample with a high level of M-protein, and the de novo sequence, which may have some level of ambiguity, provides confirmation that the peptide originates from an immunoglobulin related protein.

Using peptides extracted from de novo sequencing data, M-protein can be successfully monitored during the disease course for individual patients. Comparison of the results of the methods of the present invention with the earlier SPE results of the archived SPE gels, indicates superior sensitivity of the MS assay here presented.

Another advantage that becomes apparent from the Examples is that, for the first time, results are shown from longitudinal MM samples (tracking the same subject at different points in time) during long-term follow-up. Prospective sample cohorts may take years to collect. Using M-spike bands from archived SPE gels, from clinical laboratories, as starting material for MS analysis now enables the performance of future treatment efficacy studies with retrospective sample analysis. In the relatively small sample cohort presented in Example 1, it was shown that the methods of the present invention allow the detection of disease progression 340 days earlier, on average, than SPE analysis. The data show that the high sensitivity of MS could aid in early recognition of disease progression, monitoring response to treatment and monitoring deep responses in serum-based samples in multiple myeloma.

Sensitivity is not the only advantage of MS/PE analysis over SPE only analysis. M-spike in the beta region is a diagnostic problem for M-protein analysis by SPE. Quantification of the M-protein is based on densitometry, and because of the presence of other serum proteins that migrate in the beta region, M-protein levels cannot be determined. MS avoids this issue by targeting peptides specific for the patient M-protein, without interference from other peptides in the digested sample. Similarly, the method is not affected by the presence of therapeutic monoclonal antibodies, such as daratumumab, which is being increasingly used in treatment of MM patients.

Data was also analyzed in regards to Ig and M-protein behavior during the disease course. Levels of peptides from the constant regions of heavy and light Ig chains, as expected, appear to rise and fall during the disease course, depending on the Ig type of the M-protein. Monoclonal plasma cells expand at the expense of other cells in the bone marrow, causing the levels of other Igs, that are not the M-protein, to fall. This is referred to as immunoparesis and it has been associated with poorer survival of MM patients. MS could be a sensitive approach to monitor Igs during follow-up of MM patients. Conversely, patients suffering from MM will have an increase in a particular class of immunoglobulin. For example, one patient (patient 1 in Example 1), who has an IgA lambda M-protein, shows that peptides from the constant regions of IgA heavy chain and lambda light chain exactly follow the rise and fall pattern of the M-protein.

The strength of the MS assay of the present invention is the richness of the data. Shotgun MS approach, that measures all peptides in the digested sample, enables monitoring patient-specific M-protein peptides, but also peptides from the constant regions of Igs. In addition, peptides of special interest can be measured in a targeted way to acquire more information about M-protein behavior in the sample.

The present inventors show that it is possible to monitored the M-protein and Igs using a combination of de novo sequencing and high resolution (e.g. Orbitrap) MS in serum-based samples from MM patients, without bone marrow derived data. The methods of the present invention now allow early relapse detection and the efficacy of intervention strategies can now be investigated during clinical trials. In addition to higher sensitivity, the added value of the methods of the present invention for MM follow-up enables more frequent monitoring and provides new opportunities to evaluate cancer behavior.

### Examples

### Example 1: M-Protein Monitoring Using Gel-Based Serum Protein Electrophoresis and De Novo Sequencing by High-Resolution Mass Spectrometry

### Multiple Myeloma Patient Material

Material used in this study are archival diagnostic SPE gels from MM patients. For SPE analysis, MM patient serum has been processed and used for diagnostic purposes. The archived SPE gels were stored as dried agarose gels supported by a plastic carrier, at room temperature in a dry surrounding. As the SPE gel material is available at the archive of the Department of Clinical Chemistry, Erasmus MC, we can retrospectively select MM patients with SPE gels available for the majority/complete course of the disease.

MM patients (n=9) were retrospectively selected based on the data in the laboratory information system. Patients were selected to have SPE detectable M-protein (>1 g/L by densitometry), followed by at least one period of remission, characterized by the absence of M-protein by SPE and immunofixation electrophoresis (IFE), and at least one episode of relapse with detectable M-protein (>1 g/L by densitometry). From these patients no M-protein DNA/RNA sequence data was available. Patient samples and clinical data were coded as specified in the Dutch code of conduct for biomedical research (MEC-2019-0342). Table 1 describes the patient characteristics, the M-protein class and number of samples measured; where every sample is a time point in the disease course.

One extra patient was included outside of the mentioned selection criteria. This patient, called the reference patient, was selected because of available M-protein heavy chain DNA sequencing data and was described previously by our research group (Zajec et al. J Proteome Res. 2018; 17(3):1326-33). Serum collected from the reference patient at disease diagnosis (16.6 g/L of M-protein, defined by densitometry) was used to prepare a dilution series that was analyzed using SPE, with a total of seven dilutions with 5 fold dilution steps (M-protein concentration ranging from 3.3 g/L to 0.2 mg/L in the dilution series).

### Excision and Sample Preparation of M-Protein Bands

Cutting the M-protein bands, of the retrospectively selected MM patients, was performed in steps to ensure that the correct area of the gamma/beta fraction is cut during remission, when the M-protein band is not visible anymore by SPE. The distance between the albumin band and a detectable M-protein band was measured with a ruler. Also, the width of the M-protein band was measured. Distance from the albumin band and the width of the M-protein band were further used as markers for time points in the disease course of a patient when M-protein was not detectable by SPE. Then, each gel lane in the disease course of a patient was cut out of the SPE gel. According to the markers, M-protein band was cut out with a scalpel. Dilution series of the reference patient was cut as described above.

Each band was digested separately in a 1.5 ml Eppendorf tube. First, the bands were washed with water, 50% acetonitrile (ACN), 100% ACN and 50% ACN in 100 mM ammonium bicarbonate (ABC), then dried in Savant SC210A SpeedVac concentrator (Thermo Fisher, Munich, Germany) for 5 minutes. Subsequently, reduction and alkylation were performed using 10 mM dithiothreitol, with incubation at 56° C. for 45 minutes, and 55 mM iodoacetamide with incubation at room temperature in dark for 30 minutes. Afterwards, gel bands were washed again with water, 50% ACN, 100% ACN and 50% ACN in 100 mM ABC and dried in SpeedVac. Then, RapiGest SF (Waters, Milford, Mass.) 0.1% solution in 50 mM ABC was added to the gel bands and incubated at 37° C. for 10 minutes. After drying the gel bands in SpeedVac, 600 ng of gold-grade trypsin (Promega, Madison, Wis.) in 50 mM ABC was added to each sample and incubated at 4° C. for 5 minutes. Trypsin solution was collected in a separate tube and 50 mM ABC was added to the samples and incubated at 37° C. overnight. Tryptic peptides were extracted once with 1% trifluoroacetic acid (TFA) and twice with 0.1% TFA in 50% ACN. All extracts and trypsin solution were combined and completely dried in the SpeedVac. Tryptic peptides were resuspended in 25 μL of 0.1% TFA. All samples were cleaned-up with C18 ZipTips (Millipore, Burlington, Mass.), according to manufacturer's protocol.

### Liquid Chromatography-Mass Spectrometry Measurements

Liquid chromatography was carried out on a nano-LC system (Ultimate 3000, Thermo Fisher Scientific, Munich, Germany). Sample volumes of 10 μL were injected and separated on a C18 column (C18 Acclaim PepMap, 75 μm ID×250 mm, 2 μm particle and 100 Å pore size; Thermo Fisher Scientific, Munich, Germany). Peptides were eluted with the following binary linear gradient of buffer A and B: 4%-38% solvent B in 30 minutes. Solvent A consists of 0.1% aqueous formic acid in water and solvent B consists of 80% acetonitrile and 0.08% aqueous formic acid.

### Shotgun MS Measurements

Shotgun measurements were performed on a Q Exactive HF Orbitrap mass spectrometer (Thermo Fisher Scientific, Bremen, Germany). High resolution full scan MS data were obtained using following parameters: resolution of 60,000, AGC target of 3e6, maximum injection time of 60 ms and mass range of 375.00-1500.00 m/z. MS/MS spectra were obtained by HCD fragmentation applying 28% collision energy. MS/MS was performed on the top 20 most intense monoisotopic masses in the full scan spectra; resolution of 15,000, AGC target of 5e5, maximum injection time of 50 ms and quadrupole isolation width of 1.4 m/z. Precursor ions that were selected once for MS/MS analysis were excluded for a duration of 40 s.

### Targeted MS Measurements

Parallel reaction monitoring measurements were performed on a Q Exactive HF Orbitrap mass spectrometer. Targeted MS/MS spectra were obtained by HCD fragmentation applying 27% collision energy. The instrument was operated using following parameters: resolution of 30,000, AGC target of 5e5, maximum injection time of 250 ms and quadrupole isolation width of 1.4 m/z. MS/MS spectra were collected in centroid mode. All targeted measurements were unscheduled. m/z, charge states, fragment types of selected peptides are listed in Table 5.

De Novo Sequencing of the Mass Spectra

De novo sequencing was performed on shotgun and targeted MS raw data files using Peaks Studio 6.0 (Bioinformatics Solutions Inc., Waterloo, Canada) in order to acquire M-protein peptide sequences. In addition, Peaks DB database search tool was used for constant Ig peptide identification.(8) Following settings were used: enzyme trypsin, Orbitrap instrument and HCD fragmentation, methionine oxidation and carbamidomethylation were selected as variable and fixed post-translational modifications, respectively. For de novo analysis parent ion tolerance of 5 ppm and fragment ion tolerance of 0.02 Da were allowed. For Peaks DB analysis parent ion tolerance of 10 ppm and fragment ion tolerance of 0.02 Da were allowed and UniProt database was selected.

Selecting Patient-Specific M-Protein Peptides from De Novo Sequencing Data

First time points in the disease course, of each patient, were measured in order to select patient-specific M-protein peptides with the most correct de novo sequence. Peptide candidates have to fulfill two criteria. First, patient-specific M-protein peptide candidate has to be high abundant in a certain patient and absent or low abundant in other patient samples. Progenesis QI (Waters, Milford, Mass.) was used for label-free quantitation of MS data. Shotgun MS raw data of the first time point of each patient were imported into Progenesis with selection criteria for peptide ions: charge between 2+ and 4+, and at least two isotopes. Peptide raw abundances were exported for further filtering, for which Microsoft Excel 2010 (Microsoft, Redmond, Wash.) was used. Criteria for peptide filtering: peptide is high abundant for a certain patient and absent or low abundant (<1%) in other patients, peptide has an identification score above 7.

Second, sequence of the peptide candidate has to resemble an Ig germline sequence. Searching for Ig-looking peptides was performed using the IgBlast tool (www.ncbi.nlm.nih.gov/igblast/). If there was no resemblance to Ig germline sequences, proposed by the IgBlast tool, for a certain de novo selected peptide sequence, that peptide was rejected as an M-protein peptide candidate. All selected peptide candidate sequences had identity percentages above 68%.

### Longitudinal Monitoring of the M-Protein and Immunoglobulins

Patient-specific M-protein peptides, acquired from de novo sequencing of the shotgun MS data, were used to monitor M-protein during the disease course of each patient. All samples in the disease course were measured with targeted MS, using the selected patient-specific M-protein peptides. Targeted MS data was analyzed in Skyline(9), and the maximum height of the peaks corresponding to the patient-specific M-protein peptides were plotted against SPE M-protein (g/L) data, from the archive, for every time point in the disease course.

Peptides from the constant regions of heavy (IgG, IgA, IgM) and light (kappa and lambda) Ig chains, identified using the Peaks DB search, were used to monitor Igs during the disease courses of MM patients. We have used a subset of the peptides selected by VanDuijn et al, listed in Table 6 (VanDuijn et al. Anal Chem. 2015; 87(16):8268-74)

### Results

At least one patient-specific M-protein peptide was identified for all nine retrospectively selected MM patients using de novo sequencing by high-resolution MS (Table 2), showing 100% applicability of the method on the selected sample set. M-spike from the SPE gel was cut out and used as the starting material for MS analysis and the patient-specific M-protein peptides were identified from both the beta and the gamma region of the SPE gel. Peptide from the heavy chain was identified for four patients, peptide from the light chain for one patient, and patient-specific M-protein peptides from both heavy and light chain were identified for four patients. All peptides identified have average local confidence (ALC) scores from Peaks software above 60% for sequence fidelity.

De novo sequencing of the reference patient, with M-protein heavy chain Sanger sequencing data available, was performed the same way as for the retrospectively selected patients. However, the DNA sequence was initially blinded and only revealed for comparison after the patient-specific M-protein peptides were selected from de novo sequencing. De novo sequencing was successful, it has identified three patient-specific M-protein peptides; two from the heavy chain and one from the light chain of the M-protein (Table 3). Only peptides from the heavy chain could be compared to the DNA sequencing results, since DNA sequencing was not available for the light chain. Heavy chain peptide GLEWVSYLSSGGGSTYYADSVK, sequenced using de novo sequencing, differs from the DNA sequence, GLEWVSYISSGGGSTYYADSVK, only in amino acids leucine (L) in de novo sequence and isoleucine (I) in DNA sequence (8th position form the N terminus). As leucine and isoleucine are isobaric amino acids, with the same mass but different molecular formula, these are rarely distinguished by mass spectrometry; Peaks software reports a leucine by default for the mass corresponding to both leucine and isoleucine.

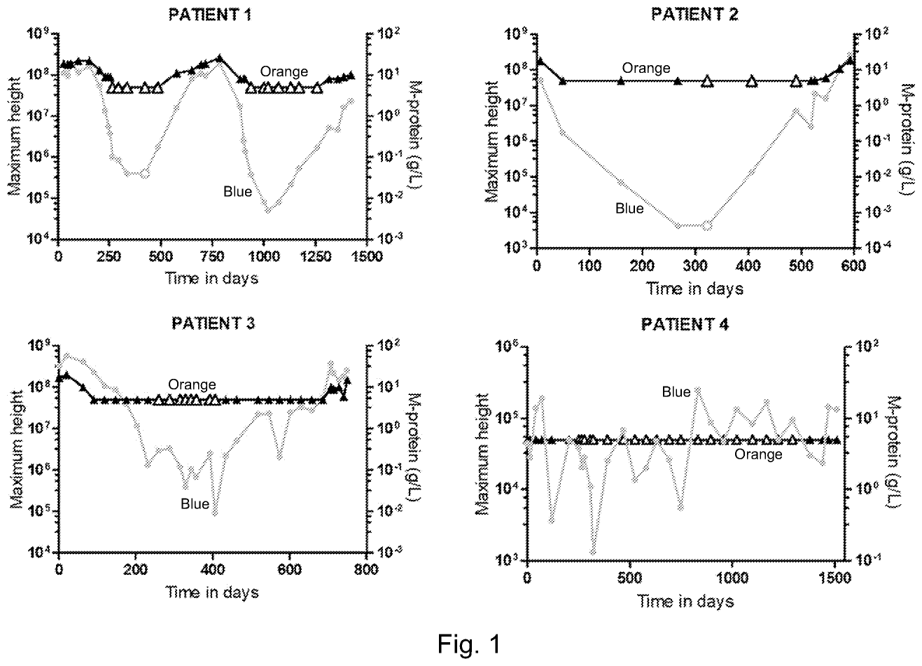

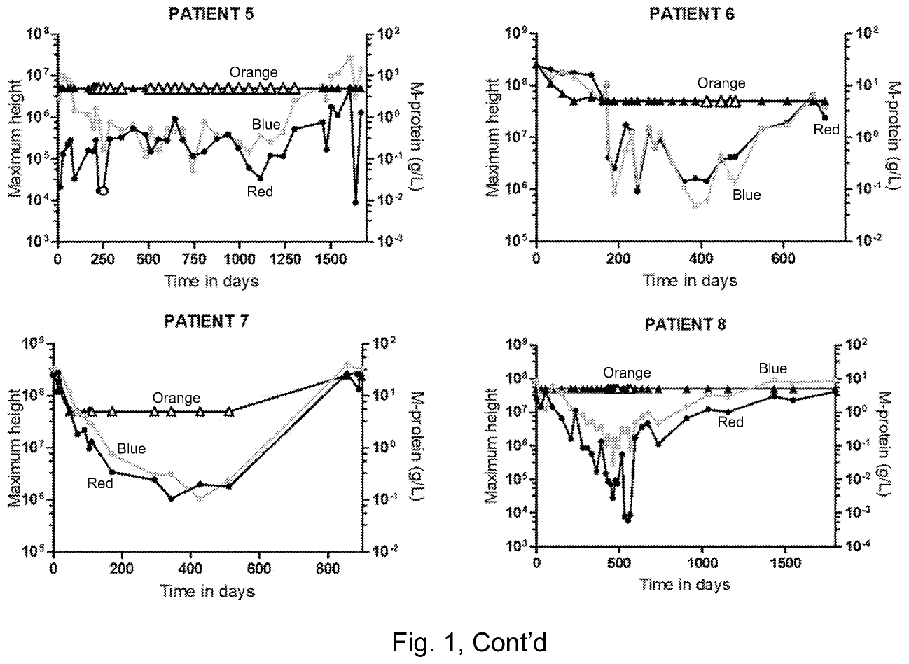

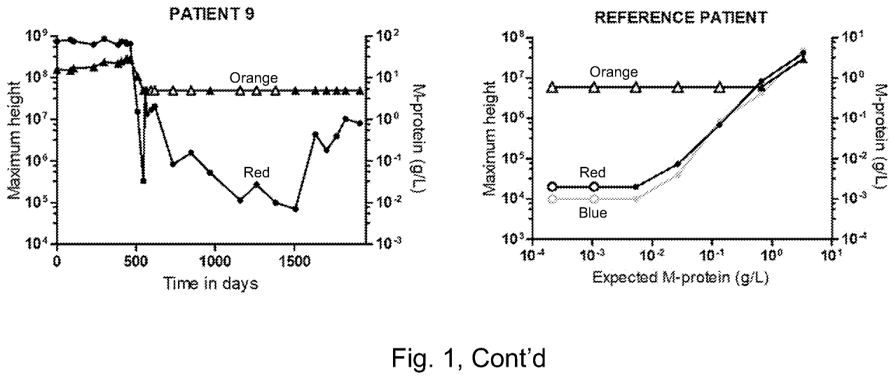

Patient-specific M-protein peptides, identified using de novo sequencing, were used as surrogates for M-protein monitoring during the disease course of each patient. Targeted MS was used to measure selected peptides and compare performance with the SPE (FIG. 1.). Results show the superiority of the MS assay over the SPE analysis in terms of sensitivity and monitoring the M-protein levels. M-spike from the dilution series of the reference patient on SPE was cut and measured by MS. Results show that MS can detect as low as 5 mg/L of the M-protein by monitoring patient-specific M-protein peptide derived from de novo sequencing data. On the other hand, no M-spike on the SPE gel was detected when the concentration of the M-protein was less than 664 mg/L in the serum sample. We have also compared time to disease progression when monitoring multiple myeloma patients with SPE and with de novo MS assay (Table 4). Progressive disease (PD), in the International Myeloma Working Group (IMWG) guidelines (Kumar et al., Lancet Oncol. 2016; 17(8):e328-e46.), is defined as 25% increase from lowest confirmed response; for SPE that is 25% increase in M-protein concentration. As the guidelines do not focus on the MS assay, we have defined PD as 25% increase in the intensity of the peak corresponding to the patient-specific M-protein peptide. With these criteria, disease progression monitored using the MS assay can be detected, on average, 340 days before SPE.

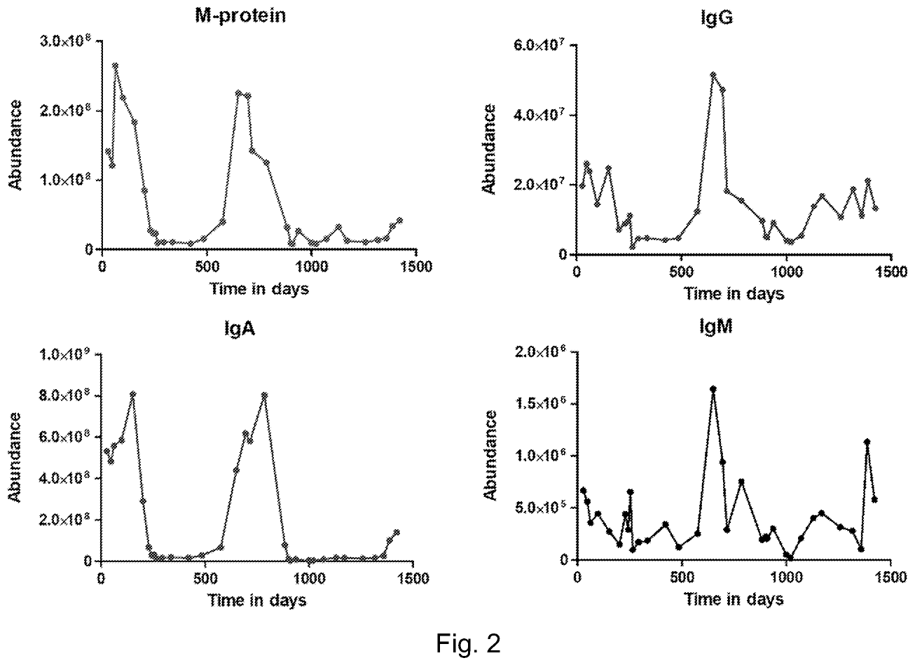

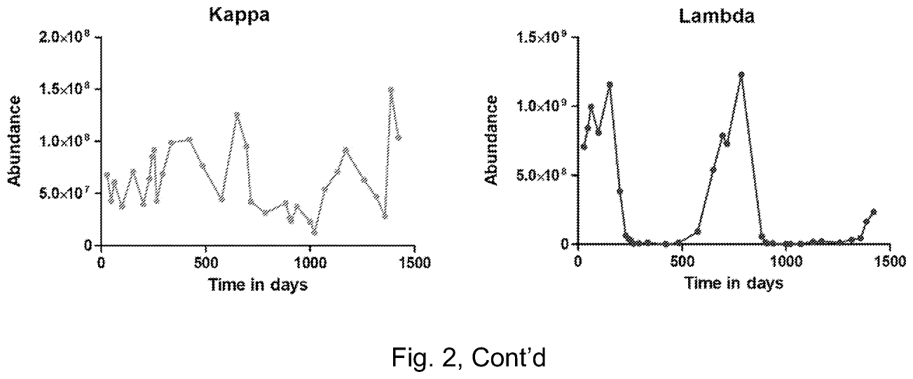

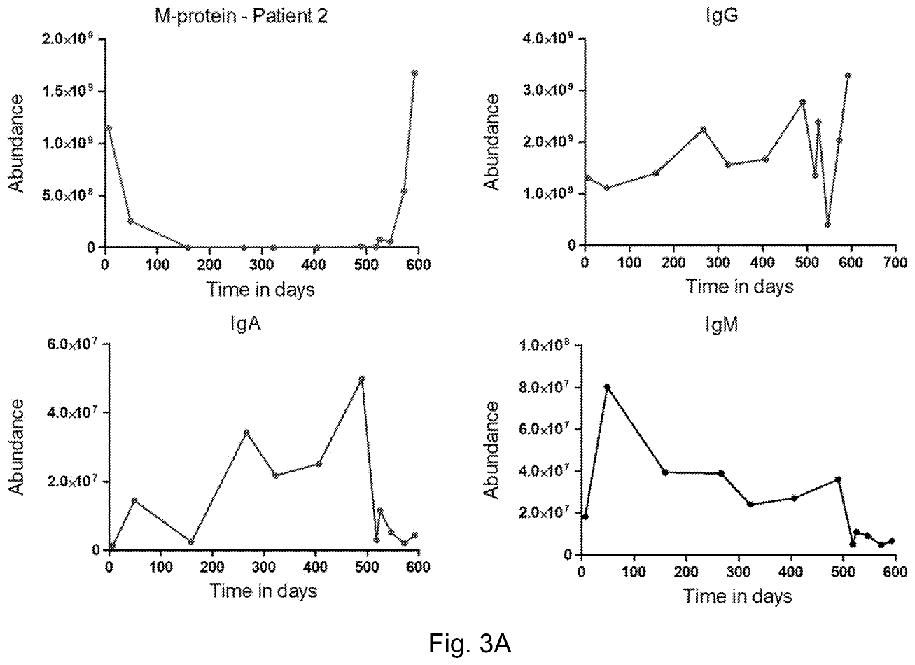

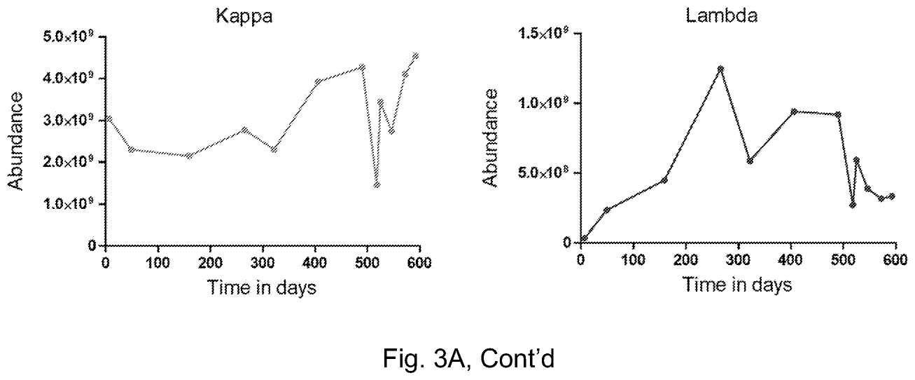

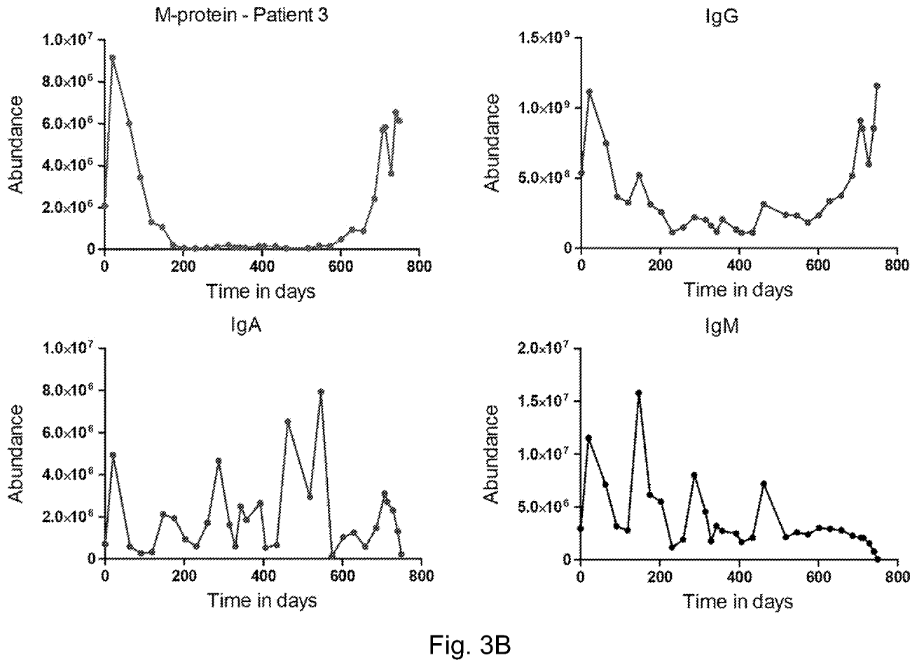

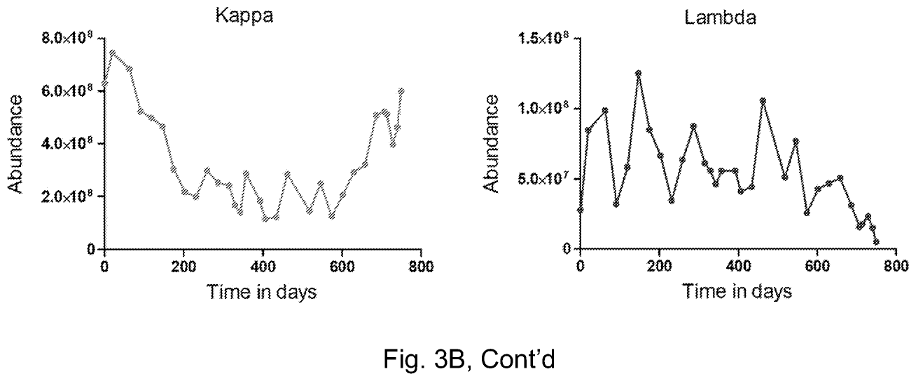

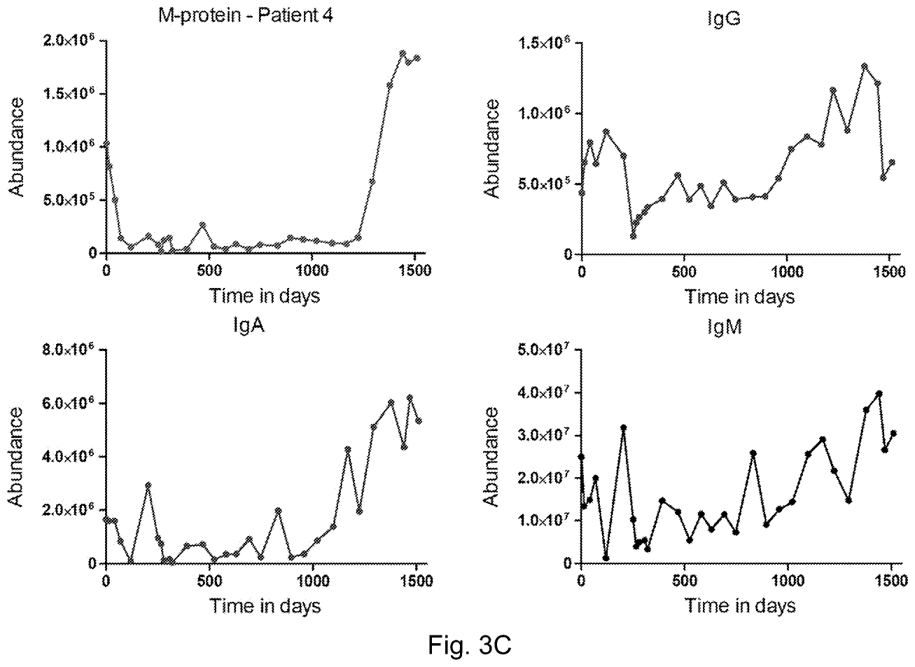

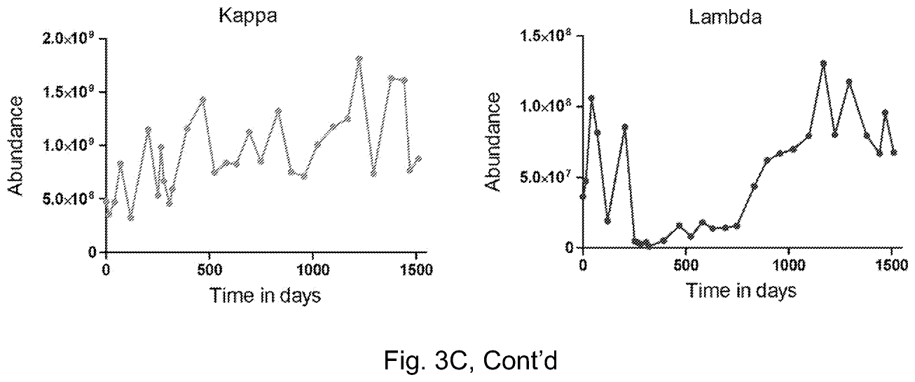

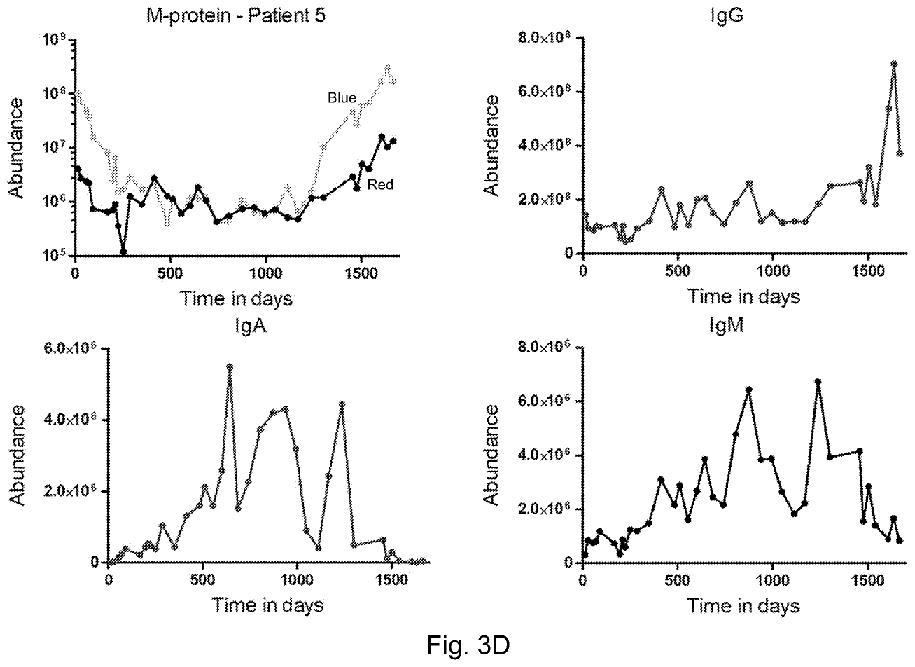

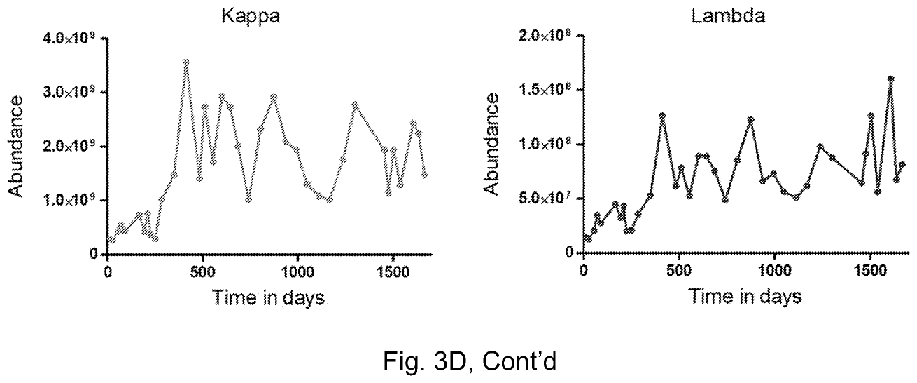

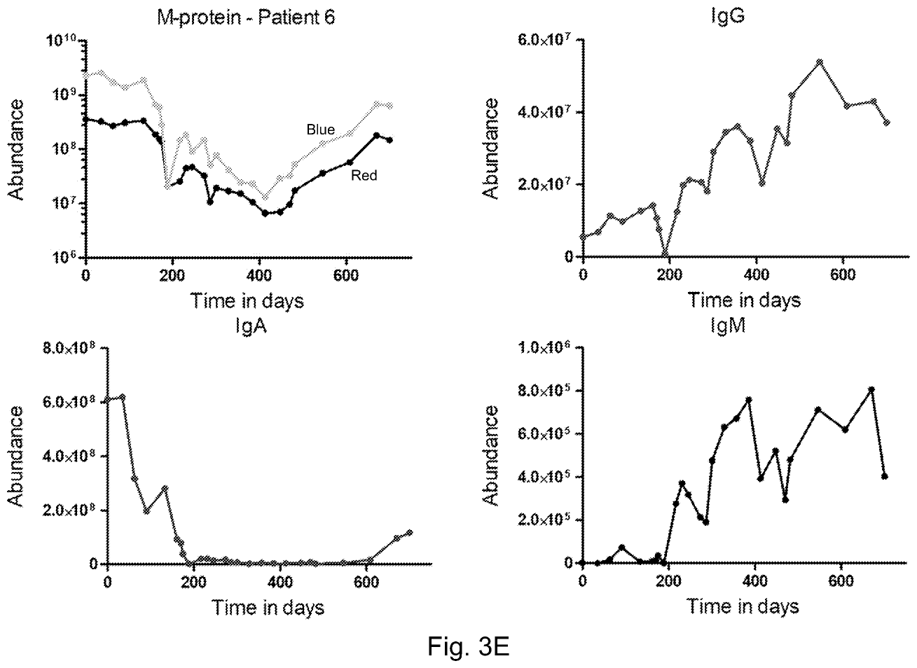

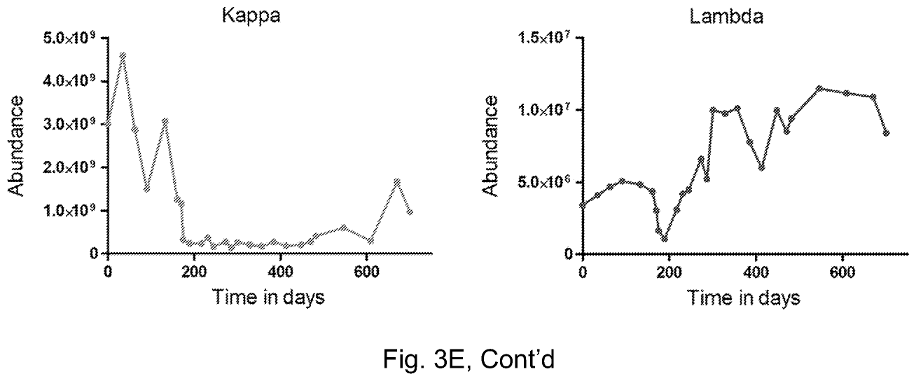

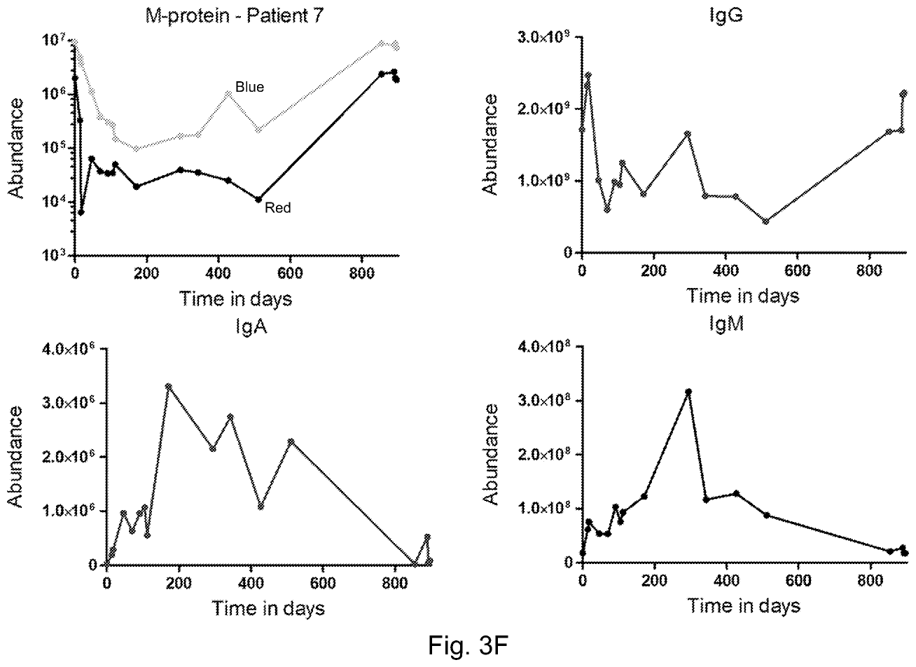

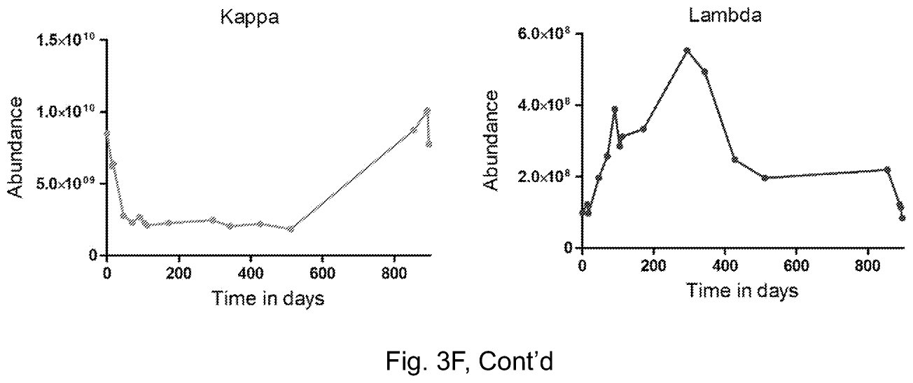

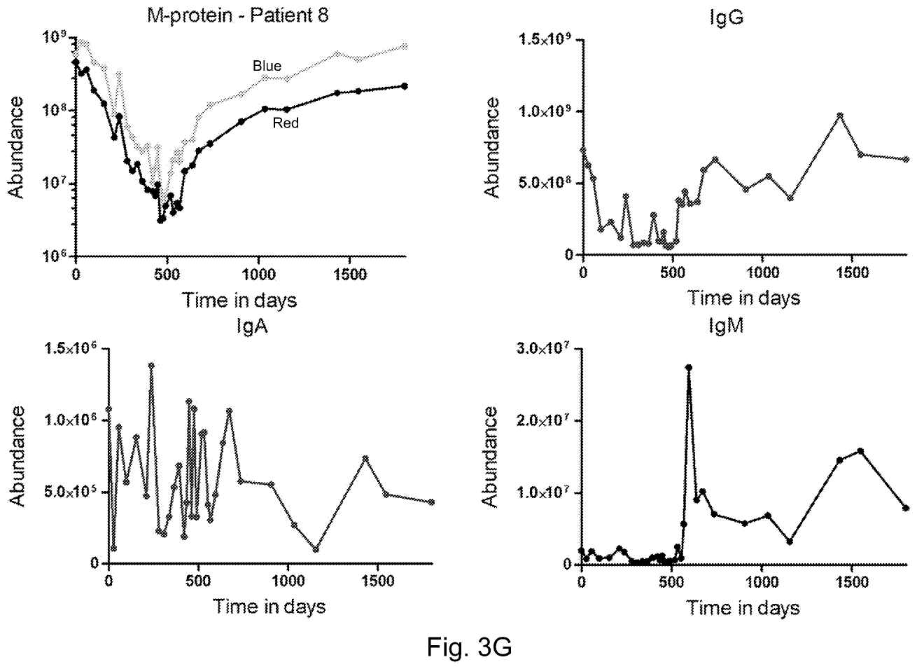

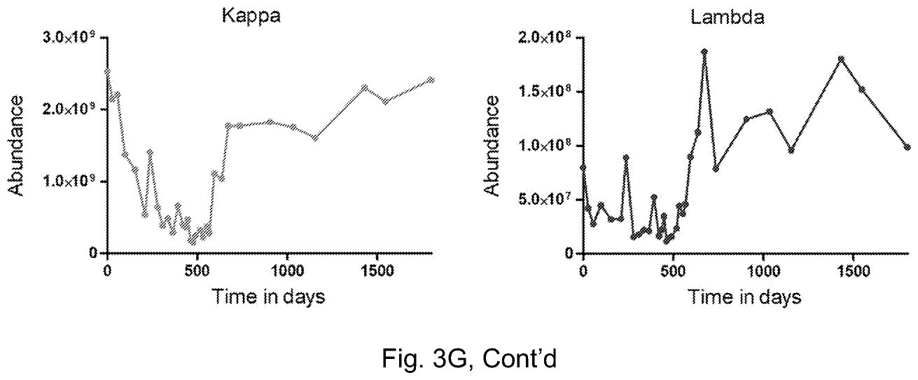

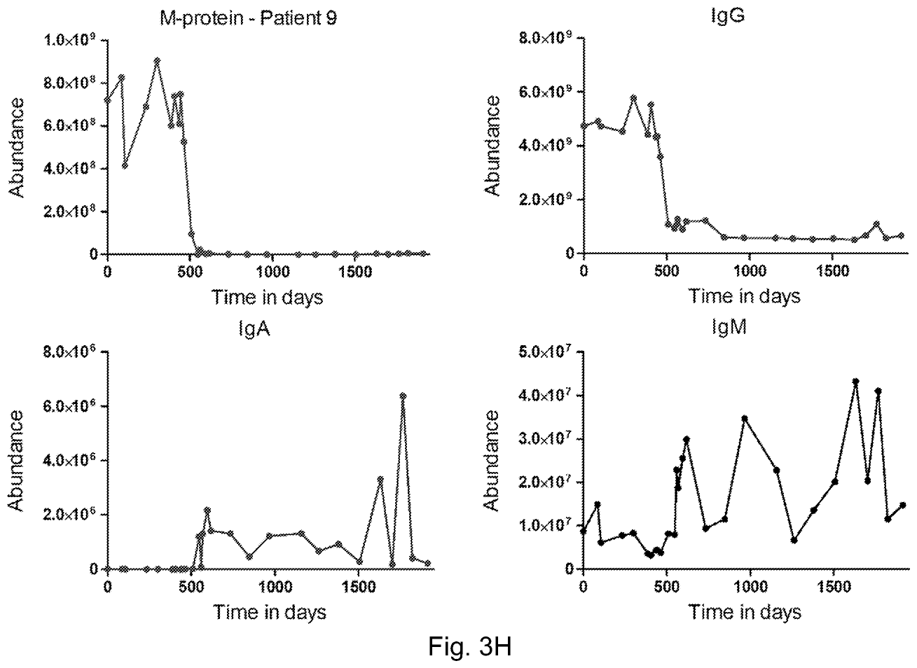

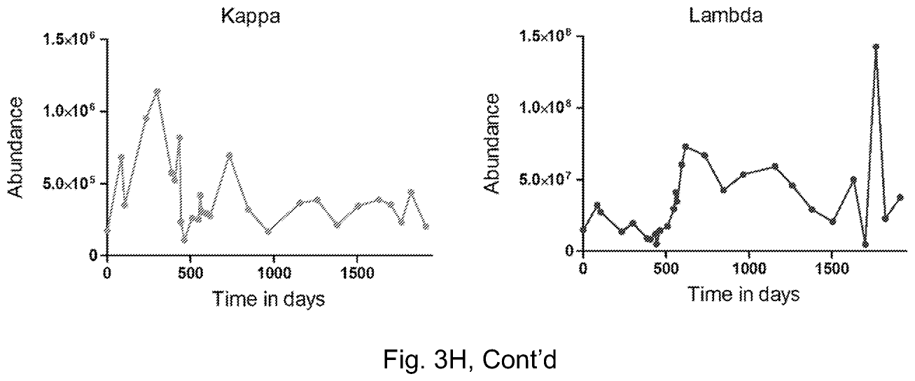

Selected peptides from the constant regions of IgG, IgA, IgM heavy chains and kappa and lambda light chains were identified in shotgun MS data from all nine retrospectively selected patients. Monitoring peptides from the constant regions of heavy and light Ig chains, from shotgun MS data, enables following changes in Ig levels during the disease course. Results for patient 1 are shown in FIG. 2.; and results for other patients are in FIG. 3A-H.

TABLE 1
Retrospectively selected multiple myeloma patient characteristics

| | | | | Samples |
|---|---|---|---|---|
| Patient | | SPE | Days | measured by |
| number | M-protein | fraction | follow-up^{a} | MS^{b} |
| #1 | IgA lambda | beta | 1423 | 33 |
| #2 | IgG kappa | gamma | 592 | 12 |
| #3 | IgG kappa | gamma | 749 | 31 |
| #4 | IgA lambda | gamma | 1511 | 30 |
| #5 | IgG kappa | gamma | 1666 | 36 |
| #6 | IgA kappa | gamma | 700 | 26 |
| #7 | IgG kappa | gamma | 896 | 17 |
| #8 | IgG kappa | gamma | 1798 | 32 |
| #9 | IgG kappa | gamma | 1913 | 28 |

^{a}number of days between the first and last time point in the disease, measured by mass spectrometry (MS);
^{b}each sample represents serum of a distinct time point in the disease course, analyzed by serum protein electrophoresis (SPE) and excised for MS measurement, as described herein in the method section.

TABLE 2
De novo results for the retrospectively selected multiple myeloma patients

| | M- | | | |
|---|---|---|---|---|
| Patient | protein | De novo M-protein peptide | ALC^{b} | Germline peptide |
| no. | chain | sequence | % | sequence |
| #1 | heavy | NSVFLEMNSLR | 79 | NSLYLQMNSLR |
| #2 | heavy | VSCTASGFSSDTHPMSWVR | 80 | LSCTASGFTFGYYPMSWVR |
| | | VSCTASGFSSDT [symbol] MSWVR | 92 | |
| | | (targeted)^{a} | | |
| #3 | heavy | LSCEASGFTFR | 90 | LSCSASGFTF |
| #4 | heavy | SPPVSVSHVEANSPGQTASLTCSGDK | 66 | PSVSVS------ |
| | | | | PGQTASITCSGDK |
| #5 | heavy | MTNMDPVDTATYYCAR | 83 | MTNMDPVDTATYYCAR |
| | light | LVLTQSPATLSASPNAAK | 63 | IVLTQSPATLSLSP |
| | | LVLTQS [symbol] TLS [symbol] S [symbol] (targeted) | 63 | |
| #6 | heavy | VELLVESGGDLVQPGGSLR | 82 | LVESGGGLVQPGGSLR |
| | light | LEVLTQSPGTLSLSPDAR | 78 | VLTQSPGTLSLSPGER |
| #7 | heavy | EDTALFYCVK | 85 | EDTAVYYCVK |
| | light | DLQMTQSPSSLSASVGDK | 64 | DIQMTQSPSSLSASVGDR |
| #8 | heavy | SMTAADTGVYYCAR | 86 | SVTAVDTGVYYCAR |
| | light | QLDLTQSPSSLSASVGDR | 76 | IQLTQSPSSLSASVGDR |
| #9 | light | LNSAGYLSLQGGVPSK | 80 | SLQSGVPSK |
| | | L [symbol] G [symbol] LQGGVPSK (targeted) | 88 | LLIYSASNLQSGVPSR |

^{a}de novo sequencing from targeted measurement data, which has a better signal to noise ratio, gave a different peptide sequence than initial shotgun measurement data, differences are bold and underlined;
^{b}ALC = average local confidence score from PEAKS.

TABLE 3
DNA and de novo sequencing results of the reference patient

| M-protein | DNA M-protein peptide | De novo M-protein | ALC^{b} | Germline peptide |
|---|---|---|---|---|
| chain | sequence | peptide sequence^{a} | % | sequence |
| heavy | GLEWVSYISSGGGSTYYADS | GLEWVSY [symbol] SSGGGSTYY | 84 | GLEWVSAISGSGGS |
| | VK | ADSVK | | TYYADSVK |
| heavy | AEDTAVYYCVR | EDTA [symbol] VYYCVR | 70 | EDTA-VYYCVR |
| light | na | ASGAALLTYLNWYQQTP | 78 | SISSYLNWYQQKPG |
| | | GK | | K |

^{a}differences between DNA and shotgun de novo sequencing are bold and underlined
^{b}ALC = average local confidence score from PEAKS
na = not available, DNA sequence information was not available for the light chain

TABLE 4
Difference in days to progressive disease (PD), when monitoring M-
protein in retrospectively selected multiple myeloma patients, with
serum protein electrophoresis (SPE) and mass spectrometry (MS).

| | | | Earlier |
|---|---|---|---|
| | | | detection |
| Patient | Days to PD^{a} − | Days to PD − | by MS vs |
| number | SPE^{b} | MS^{c} | SPE (days) |
| #1 | 576, 1317^{d} | 485, 1071 | 82, 246 |
| #2 | 546 | 406 | 140 |
| #3 | 707 | 434 | 273 |
| #4 | 1437 | 832 | 605 |
| #5 | 1424 | 1141 | 283 |
| #6 | 700 | 448 | 252 |
| #7 | 854 | 511 | 343 |
| #8 | 1798 | 905 | 893 |
| #9 | 1913 | 1631 | 282 |

^{a}progressive disease, in the International Myeloma Working Group (IMWG) guidelines(5), is defined as 25% increase from lowest confirmed response, for SPE M-protein lower or equal to 5 g/L an increase of 1 g/L is defined as progressive disease;
^{b}in case of stable disease (no increase from 5 g/L) day of the last measurement in the disease course was taken (patients 6, 8 and 9);
^{c}in case of patients 5, 6, 7 and 8, with both heavy and light chain data, average was calculated if days to disease progression were different for heavy and light chain;
^{d}patient 1 has two periods of remission (M-protein is not detectable by SPE), therefore two occurrences of disease progression.

TABLE 5
Sequence, m/z, charge states and fragment types of selected patient-
specific M-protein peptides used for M-protein monitoring with targeted mass
spectrometry.

| Patient | | | | Identified |
|---|---|---|---|---|
| no. | M-protein peptide sequence | m/z | z | fragment types |
| #1 | NSVFLEM[Ox]NSLR | 663.3295 | 2 | y1-9; b1-4 |
| #2 | VSC[cam]TASGFSSDTHPMSWVR | 1056.463 | 2 | y1-5, y7-12; b2-12, b14 |
| | VSC[cam]TASGFSSDTYAMSWVR | 1056.463 | 2 | y1-12; b2-9 |
| | (targeted)^{a} | | | |
| #3 | LSC[cam]EASGFTFR | 637.7953 | 2 | y1-10; b1-8 |
| #4 | SPPVSVSHVEANSPGQTASLTC[cam]SGDK | 871.4134 | 3 | y2-10, y13, y14; b2-7 |
| #5 | M[ox]TNM[ox]DPVDTATYYCAR | 970.8948 | 2 | y1-12; b1-5, b7, b8 |
| | LVLTQSPATLSASPNAAK | 884.9938 | 2 | y7-15; b2-6 |
| | LVLTQSAPTLSLSDAPR (targeted) | 884.9938 | 2 | y1, y4-9 ,711-14; b2-6 |
| #6 | VELLVESGGDLVQPGGSLR | 963.0206 | 2 | y1-15; b2-ll |
| | LEVLTQSPGTLSLSPDAR | 942.5073 | 2 | y1, y3-14; b2-7 |
| #7 | EDTALFYC[cam]VK | 623.2946 | 2 | y1-9; b2-5 |
| | DLQM[ox]TQSPSSLSASVGDK | 933.9411 | 2 | y1-14; b2-7 |
| #8 | SM[ox]TAADTGVYYC[cam]AR | 791.3372 | 2 | y1-12; b2-9, b1l |
| | QLDLTQSPSSLSASVGDR | 620.9814 | 3 | y1-14; b1-10 |
| #9 | LNSAGYLSLQGGVPSK | 795.9279 | 2 | y1-8; b2-9, b12, b13 |
| | LSLYGASNLQGGVPSK (targeted) | 795.9279 | 2 | y1-14; b2-10, b12, b13 |

ox = oxidation;
cam = carb amidomethylation;
^{a}de novo sequencing from targeted measurement data gave a different peptide sequence than shotgun measurement data.

TABLE 6
Peptides from the constant regions of heavy and light immunoglobulin
chains used to monitor immunoglobulins during the disease courses of
multiple myeloma patients.

| Protein | Accession^{a} | Peptide sequence | Peptide mass |
|---|---|---|---|
| IgG | P01860 | DTLMISR | 834.4268 |
| IgA | P01876 | YLTWASR | 895.4566 |
| IgM | P0DOX6 | VTSTLTLK | 861.5183 |
| | P01871 | YVTSAPMPEPQAPGR^{b} | 1615.7660 |
| Kappa | P01834 | TVAAPSVFIFPPSDEQLK | 1945.0185 |
| Lambda | P0DOX8 | SYSCQVTHEGSTVEK | 1710.7502 |

^{a}UniProt c atabase accession number;
^{b}for patients 6 and 7

### Example 2. Integrating Serum Protein Electrophoresis with Mass Spectrometry, a New Workflow for M-Protein Detection and Quantification

Serum protein electrophoresis (SPE) and immunofixation electrophoresis (IFE) are standard tools for multiple myeloma (MM) routine diagnostics. M-protein is a biomarker for MM that can be quantified with SPE and characterized with IFE.

It was investigated if immunoglobulin enrichment could be combined with SPE/IFE with targeted mass spectrometry (MS). It will be shown in this Example that the combination SPE-MS assay offers the possibility to detect M-protein with higher sensitivity than SPE/IFE, which leads to better analysis of minimal residual disease in clinical laboratories. In addition, analysis of archived SPE gels could be used for retrospective MM studies.

In this Example, two different approaches of measuring M-protein and therapeutic monoclonal antibodies (t-mAbs) from SPE gels are investigated. After extracting specific peptides from SPE gels, they can be quantified using stable isotope labeled (SIL) peptides and measured by Orbitrap mass spectrometry. Alternatively, to circumvent personalized SIL peptides, extracted peptides can be labeled with tandem mass tags (TMT). Both approaches are not hampered by the presence of t-mAbs.

Using SIL peptides, limit of detection of the M-protein is approximately 100-fold better than with conventional SPE/IFE. Using TMT labeling, M-protein can be measured and compared in different samples from the same patient. We have successfully measured M-protein specific peptides extracted from the SPE gels utilizing SIL peptides and TMT.

### Biological and Other Material

All MM patient and control sera, daratumumab and SIL peptides were used as described before (Zajec et al., J Proteome Res, 17, 1326-1333 (2018)). Briefly, serum of the MM patient is used for which heavy chain Sanger sequencing data is available. All serum samples and clinical data were coded and anonymized as specified in the Dutch code of conduct for biomedical research. Daratumumab was purchased from Janssen Biotech, Leiden, The Netherlands. SIL peptides (Pepscan B.V., Lelystad, The Netherlands) were used as internal standards for quantification. Peptides, from the patient M-protein and daratumumab, were considered as proteotypic peptides and candidates for stable isotope labeling if they were tryptic peptides with mutations in the amino acid sequence compared to the germline reference sequence. The stable isotope labeled peptides used, are shown in Table 7.

TABLE 7
Peptides used for quantification of daratumumab and M-protein.

| | | | | Collision | |
|---|---|---|---|---|---|
| | | | | energy | Extracted |
| Protein | Peptide sequence | m/z | z | (%) | fragments |
| daratumumab | GLEWVSAISGSGGGTYYADSVK | 735.3551 | 3 | 30 | b2, b3, y5, |
| | GLEWVSAISGSGGGTYYADSVK* | 738.0265 | | | y6, y7 |
| M-protein | GLEWVSYISSGGGSTYYADSVK | 1163.5473 | 2 | 21 | b5, b8, |
| | GLEWVSYISSGGGSTYYADSVK* | 1167.5544 | | | y14, y15 |
| | | | | | y12,y13, |

*stable isotope labeled amino acid

### Dilution Series Preparation for M-Protein and Daratumumab Measurements

To determine the sensitivity of the SPE-MS assay we have prepared a series of M-protein dilutions. MM patient serum (16.6 g/L of M-protein defined by densitometry) was diluted into a healthy control serum that did not contain M-protein or daratumumab. In this way, 8 dilutions with 5 fold incremental steps were prepared. Daratumumab was spiked at a constant concentration of 1 g/L across the dilutions. All dilutions and control serum were measured by SPE. Highest concentration M-protein sample was measured in the first and last SPE lane. By diluting the MM patient serum into a control serum we dilute the M-protein but keep the background proteins at a constant level. That way we can mimic the decreasing tumor load. Dilution series was prepared, cut, digested and measured in duplicate.

To estimate the difference in sensitivity between SPE and IFE as starting materials for mass spectrometry analysis, daratumumab was diluted into a control serum and measured on both SPE and IFE. Two dilutions were prepared: 1 g/L and 0.04 g/L and measured in triplicate; we have measured SPE M-protein band and IFE M-protein band from the IgG lane.

SPE and IFE were performed on a Hydrasys device (Sebia, Evry, France) using reagents from Sebia, according to manufacturer's protocols. Briefly, for SPE 10 μL of serum is applied and allowed to diffuse into the gel at room temperature for 30 seconds; for IFE 10 μL of six times diluted serum is applied and allowed to diffuse into the gel for 60 seconds.

### Cutting and Digesting SPE and IFE Gel Bands for Proteomic Analysis

The gel bands were marked with a ruler and a scalpel; and were cut with scissors. To assure that the bands that are cut from the gels are approximately the same size, ruler was placed directly above and below the band and straight lines with a scalpel were cut in the gel. That straight line was then cut with the scissors, to reduce plastic particles. The gel bands of interest were cut with scissors assuring minimum of plastic around the band. Each band was digested separately in a 1.5 ml Eppendorf tube.

Gel pieces were washed with water, 50% acetonitrile (ACN), 100% ACN and 50% ACN in 100 mM ammonium bicarbonate, then dried in Savant SC210A SpeedVac concentrator (Thermo Fisher, Munich, Germany) for 5 minutes. RapiGest SF (Waters, Milford, Mass.) 0.1% solution in 50 mM ABC was added to the gel samples and incubated at 37° C. for 10 minutes. After drying the gel pieces again in the SpeedVac for 5 minutes, 600 ng of gold-grade trypsin (Promega, Madison, Wis.) was added to each sample and incubated at 4° C. for 5 minutes. Trypsin solution was collected in a separate tube and 50 mM ABC was added to the samples and incubated at 37° C. overnight. Tryptic peptides were extracted once with 1% TFA and twice with 0.1% TFA in 50% ACN. All extracts and trypsin solution were combined and completely dried in the SpeedVac. Tryptic peptides from the M-protein and daratumumab were resuspended in 25 μL of 10 fmol/μL SIL peptide solution in 0.1% TFA.

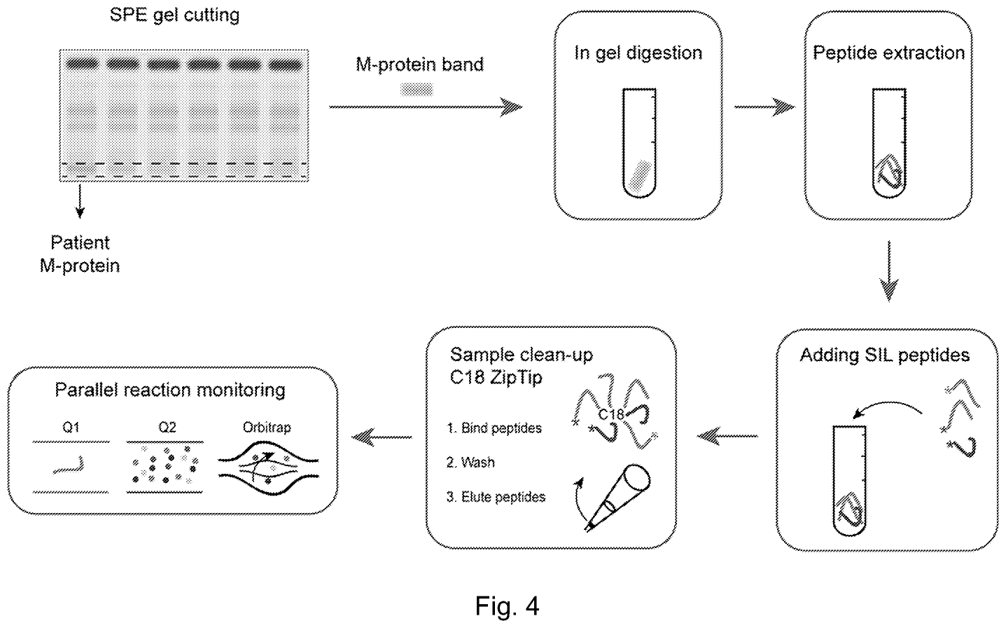

Before continuing with the LC-MS analysis all samples were cleaned-up with C18 ZipTips (Millipore, Burlington, Mass.) in order to preventively remove any particles coming from the plastic gel carrier or dried agarose. ZipTips were used according to manufacturer's protocol. Briefly, after peptide binding to the C18 material, the ZipTip pipette tip is washed with 0.1% TFA in Mili-Q water, and the peptides are eluted with 0.1% TFA/50% ACN. FIG. 4. shows the experimental design for the targeted mass spectrometry workflow using SPE gel as starting material and SIL peptides for quantification.

### Liquid Chromatography-Mass Spectrometry (LC-MS) Measurements

Liquid chromatography was carried out on a nano-LC system (Ultimate 3000, Thermo Fisher Scientific, Munich, Germany). Samples were separated on a C18 column (C18 PepMap, 75 μm ID×250 mm, 2 μm particle and 100 Å pore size; Thermo Fisher Scientific, Munich, Germany) and peptides were eluted with the following binary linear gradient of buffer A and B: 4%-38% solvent B in 30 minutes. Solvent A consists of 0.1% aqueous formic acid in water and solvent B consists of 80% acetonitrile and 0.08% aqueous formic acid.

Parallel reaction monitoring (PRM) measurements were performed on Q Exactive HF Orbitrap mass spectrometer (Thermo Fisher Scientific, Bremen, Germany). For electrospray ionization, nano ESI emitters (New Objective, Woburn, Mass.) were used and a spray voltage of 1.7 kV was applied. For daratumumab and M-protein peptides the instrument was operated in a targeted MS/MS using following parameters: quadrupole isolation width of 4.0 m/z, HCD fragmentation, MS/MS AGC target of 500,000 at a maximum injection time of 250 ms, and Orbitrap resolution of 30,000. MS/MS spectra were collected in centroid mode. m/z, charge states, collision energies and fragment types for quantification of daratumumab and M-protein are listed in Table 7.

### Data Processing

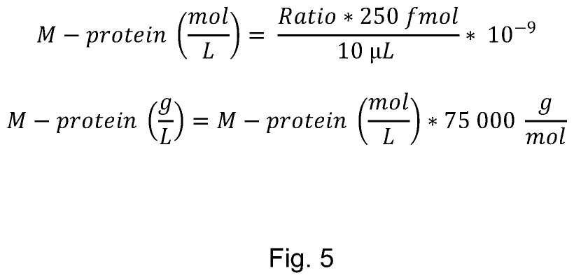

Signals were integrated using Skyline (MacLean et al., Bioinformatics, 26, 966-968 (2010)). Concentration of each peptide was calculated from the peak area ratio between the endogenous and the SIL peptides. Calculation of M-protein concentration from PRM data is shown in FIG. 5. Limit of detection (LOD) for the M-protein was defined as (3.3×SD)/slope, and lower limit of quantification (LLoQ) as 3×LOD, according to ICH guidelines (http://www.ich.org); where SD was calculated for control serum without M-protein and with low concentration of M-protein (two highest dilutions). Recovery (%) was calculated for both M-protein and daratumumab by dividing measured concentration of M-protein with expected concentration of M-protein. Expected concentration is the M-protein concentration in the serum that is loaded on the SPE.

### Tandem Mass Tag (TMT) Labeling

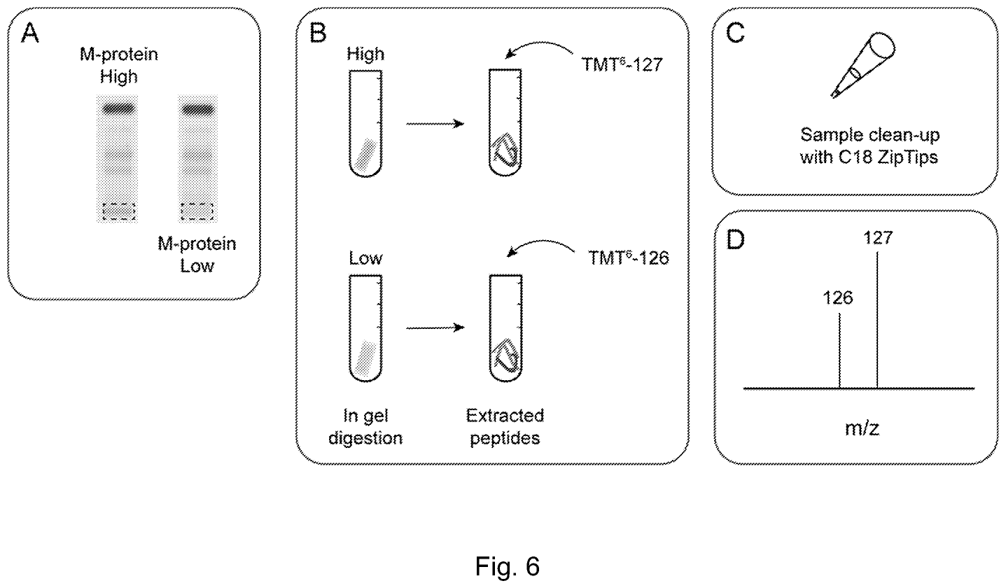

For TMT labeling SPE gels and the M-protein dilution series were prepared as described above for the SIL labeling approach. The same MM patient serum was diluted into a control serum with 1 g/L of ipilimumab (Brystol-Myers Squibb, Utrecht, The Netherlands). TMTsixplex Isobaric Label Reagent Set was purchased from Thermo Fisher Scientific. For samples labeled with TMT, digestion of the gel bands was performed without Rapigest, ABC was exchanged for triethylammonium bicarbonate (TEAB) buffer and TFA was neutralized with TEAB before adding the TMT labels to the samples. TMT labeling was performed according to manufacturer's protocols. Two samples were labeled with TMT and they will be referred to as M-protein high concentration sample (3.3 g/L of M-protein, 1 g/L of ipilimumab) and M-protein low concentration sample (5.3 mg/L of M-protein, 1 g/L of ipilimumab). M-protein high concentration sample was labeled with TMT⁶-127 label (127.1248 monoisotopic reporter mass), and M-protein low concentration sample was labeled with TMT⁶-126 (126.1277 monoisotopic reporter mass). FIG. 6. shows the experimental design for the mass spectrometry workflow using TMT for labeling the M-protein and ipilimumab specific peptides. After the labeling samples were mixed and targeted mass spectrometry analysis was performed.

Mass spectrometry measurements were performed on Orbitrap Fusion (Thermo Fisher Scientific). For ipilimumab and M-protein peptides the instrument was operated in a targeted MS/MS using following parameters: quadrupole isolation width of 1.4 m/z, HCD fragmentation, MS/MS AGC target of 50,000 at a maximum injection time of 246 ms, and Orbitrap resolution of 120,000. MS/MS spectra were collected in centroid mode. m/z, charge states and collision energies of ipilimumab and M-protein are listed in Table 8.

TABLE 8
Sequences and masses of peptides labeled with TMT, at both the N-
terminal amine and at the amine of lysine.

| | | | | Collision |
|---|---|---|---|---|
| | | | | energy |
| Protein | Peptide sequence | m/z | z | (%) |
| ipilimumab | T^{TMT}GWLGPFDYWGQGTLVTVSSASTK^{TMT} | 1006.1989 | 3 | 38 |
| M-protein | G^{TMT}LEWVSYISSGGGSTYYADSVK^{TMT} | 1392.7103 | 2 | 38 |

### Results

Removing Contamination/Particles Coming from the SPE Gel Pieces

C18 ZipTips were used as a filter to remove any particles or contamination coming from plastic gel carrier/dried agarose that might interfere with the assay. The clean-up, using C18 ZipTips, was performed successfully for all samples, and they were separated and analyzed on the LC-MS systems without excluding any sample.

### Sensitivity of the SPE-MS Assay

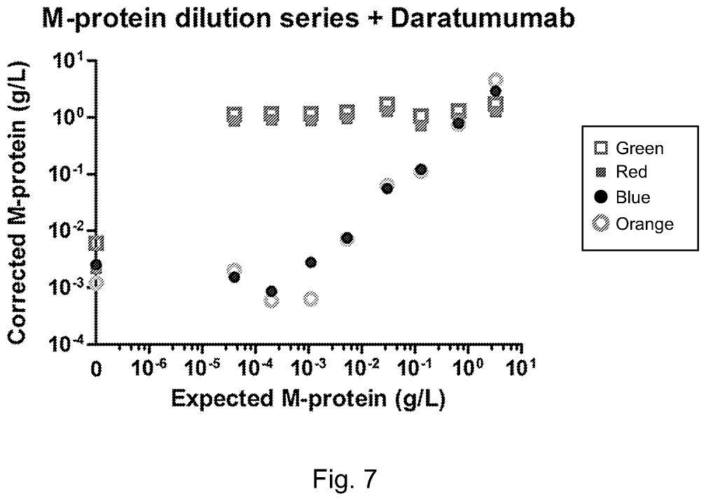

Dilution series of the M-protein were prepared using control serum, daratumumab was spiked into each sample at a fixed concentration of 1 g/L. Specific peptides for the M-protein and for daratumumab were successfully identified and quantified. Addition of daratumumab shows that two different monoclonal antibodies can be identified from the same SPE gel band without interference. LOD and LLoQ were determined for the peptide GLEWVSYISSGGGSTYYADSVK of the M-protein. LOD is 2.7 mg/L and LLoQ is 8.1 mg/L. Dilution series was measured in duplicate as shown in FIG. 7. Measurements were linear over 3 logs (r₁²=0.9951 and r₂²=0.9988 for two dilution series), until M-protein signals were at the detection limit. Recovery (with standard deviation) from the serum, that was measured on the SPE gel, was calculated for both the M-protein and daratumumab, relative to the amount of serum loaded on the gel. Recovery for M-protein is 1.34% (0.42) and it was calculated as an average of first 5 dilutions; which were above the LLoQ. For daratumumab recovery is 0.87% (0.18) and it was calculated as an average of all 8 dilutions with spiked daratumumab. The volume of serum that is applied on the SPE sample applicator is 10 μL, however because of the way the SPE is performed, it has been reported in the literature that just a small fraction of the sample is actually introduced into the gel. If just a fraction of serum enters the gel, it is evident that the amount that will be measured by mass spectrometry will be proportionally lower. We have used these 10 μL to calculate the concentration of the M-protein in serum (FIG. 5), meaning that the recovery we calculate includes the loss of serum on the SPE/IFE applicator, loss during the digestion and loss during C18 ZipTip clean-up. It is a recovery for the integrated SPE-MS workflow and as a result the numbers are relatively low.

### SPE and IFE as Starting Materials for the MS Assay

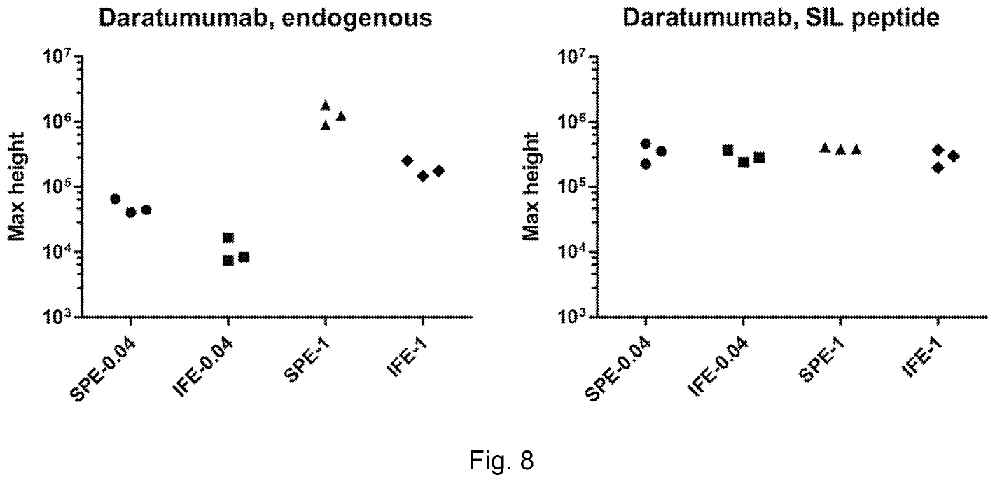

To estimate the difference in sensitivity between SPE and IFE as starting materials for mass spectrometry measurements, two dilutions of daratumumab (1 g/L and 0.04 g/L) in control serum were measured on both SPE and IFE gels. As showed in FIG. 8., maximum heights of the peaks corresponding to the endogenous peptide targeted for daratumumab (GLEWVSAISGSGGGTYYADSVK) are higher in SPE than IFE (IgG lane) samples for both dilutions. Average peak maximum is 5 times higher in SPE than in IFE for the lower concentration (0.04 g/L), and 7 times higher in SPE than in IFE for the higher concentration (1 g/L). Maximum heights of the peaks for the SIL peptide, which was added to all samples in the same concentration (10 fmol/μL), were measured as a control. Peaks for the SIL peptide do not show this difference in maximum peak height.

### TMT Labeling of MM Patient Serum

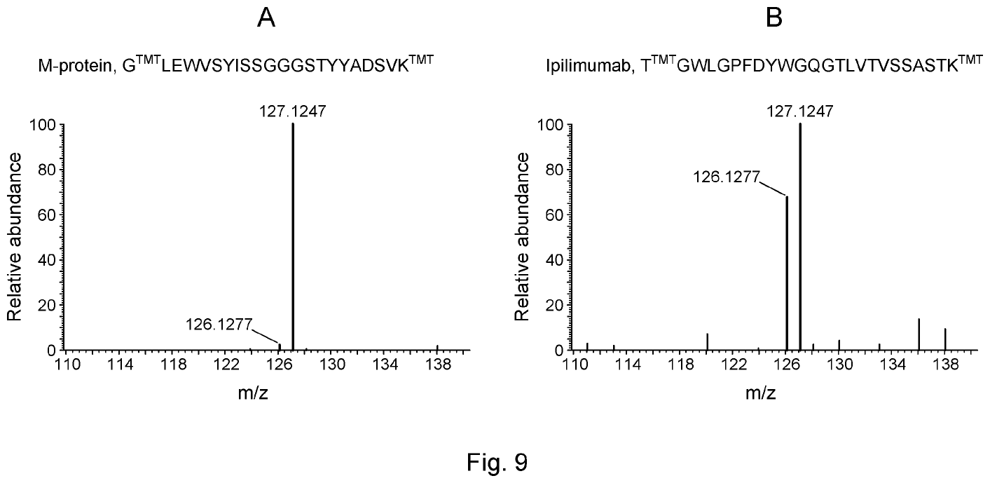

The targeted MS assay using SIL peptides requires synthesis and stable isotope labeling of specific M-protein peptides for every new patient. To explore an alternate workflow that circumvents the need for the personalized labeling approach, MM patient serum with added ipilimumab was labeled with tandem mass tags. Two samples with different M-protein concentrations, and the same ipilimumab concentration (1 g/L), were labeled with two different TMT labels, TMT⁶-127 and TMT⁶-126. After labeling the M-protein high concentration sample (3.3 g/L of M-protein) and M-protein low concentration sample (5.3 mg/L of M-protein), samples were mixed and measured. Both reporter ions can be observed in the MS² spectra, as shown in FIG. 9. We have labeled two M-protein samples as the rationale behind the high and the low concentration M-protein sample is that the initial serum sample with high tumor load and high M-protein concentration is quantified by SPE in routine clinical laboratory testing. The concentration of the M-protein is then known, therefore that initial sample can be used as a reference for follow-up samples. Samples with an M-protein concentration lower than the limit of quantification of the SPE are ideally further investigated with a more sensitive method. Concentration of the low M-protein sample can now be calculated from the ratio between the two reporter ions and known concentration of the initial serum sample. Both samples, high and low concentration of the M-protein, were successfully labeled and ipilimumab is distinguished from the M-protein.

### CONCLUSION

We have successfully digested M-protein bands from the serum protein electrophoretic gels, extracted M-protein specific peptides and removed any contamination using C18 ZipTip pipette tips. We have performed targeted analysis and quantified the M-proteins using stable isotope labels. Sensitivity of SPE-MS approach exceeds the sensitivity of serum protein electrophoresis for two orders of magnitude.

We have also explored a non-personalized labeling approach using tandem mass tags and we have succeeded to label peptides, extracted from the SPE gels, with tandem mass tags in order to compare follow-up samples from the same patient with different M-protein concentrations. This approach has the potential to allow a personalized test without personalized reagents.

1. A method for quantifying a monoclonal (M-) protein in a sample of a subject, the method comprising the steps of:
subjecting a body sample of a subject to protein electrophoresis in a gel to separate proteins comprised in said body sample into different protein fractions, optionally followed by immunofixation electrophoresis (IFE) and further optionally involving immunostaining of the gel;
drying said gel to provide a dried gel;
optionally, archiving said dried gel;
excising from said dried gel a gel part comprising, or suspected of comprising, a M-protein;
performing an enzymatic digestion on proteins present in said gel part or on a protein extract thereof in order to provide a peptide digest comprising at least one M-protein peptide;
subjecting said peptide digest comprising said at least one M-protein peptide to mass spectrometry (MS) to determine a quantity of said at least one M-protein peptide, thereby quantifying said M-protein in said sample by MS.

2. A method for quantifying a M-protein in a sample of a subject, the method comprising the steps of:
providing from a dried gel an excised gel part comprising, or suspected of comprising, a M-protein from a body sample of a subject; wherein said dried gel is obtained by performing the steps of:
subjecting a body sample of a subject to protein electrophoresis in a gel to separate proteins comprised in said body sample into different protein fractions, optionally including immunofixation electrophoresis (IFE) and further optionally including immunostaining of the gel;
drying said gel;
optionally, archiving said dried gel;
performing an enzymatic digestion on proteins present in said gel part or on a protein extract thereof in order to provide a peptide digest comprising at least one M-protein peptide;
subjecting said peptide digest comprising said at least one M-protein peptide to mass spectrometry (MS) to determine a quantity of said at least one M-protein peptide, thereby quantifying said M-protein in said sample.

3. The method according to claim 1, wherein the step of subjecting said peptide digest comprising said at least one M-protein peptide to mass spectrometry (MS) coincides or is preceded by the selection of at least one subject-specific M-protein peptide, and wherein the selection of at least one subject-specific M-protein peptide comprises the steps of:
(i) providing a body sample of said subject comprising a plasma cell producing the M-protein, determining the amino acid sequence of the M-protein produced by said plasma cell, and selecting a subject-specific M-protein peptide sequence that is predicted to be generated by said enzymatic digestion of said M-protein; or,
(ii) providing the amino acid sequence of the M-protein produced by said subject, and selecting a subject-specific M-protein peptide sequence that is predicted to be generated by said enzymatic digestion of said M-protein; or,
(iii) subjecting said peptide digest comprising said at least one M-protein peptide to MS, determining the amino acid sequence of a multitude of peptides in said peptide digest by de novo peptide sequencing, and selecting a subject-specific M-protein peptide sequence from the sequences determined,
wherein said method further comprises the step of subjecting said peptide digest comprising said at least one M-protein peptide to targeted MS to thereby quantify the selected subject-specific M-protein peptide in said peptide digest.

4. The method according to claim 3, wherein said method further comprises the steps of
preparing a stable isotope labelled (SIL) variant of said selected subject-specific M-protein peptide as a reference peptide, and
adding said reference peptide to said peptide digest after said step of enzymatic digestion;
wherein the quantity of said at least one M-protein peptide is determined by comparing an MS signal of said selected subject-specific M-protein peptide to an MS signal of said reference peptide, thereby quantifying said M-protein in said sample.

5. The method according to claim 1, wherein the subject is a patient suffering from, having suffered from, or at risk of suffering from plasma cell dyscrasia, or a patient undergoing treatment for plasma cell dyscrasia.

6. The method according to claim 1, wherein the subject is in remission of plasma cell dyscrasia, and suffering from or at risk of suffering from MRD of plasma cell dyscrasia.

7. The method according to claim 6, wherein excision of said gel part from said dried gel is performed by using a patient-specific gel cutting template in order to ensure that an M-protein band that is present but invisible by normal SPE staining is correctly excised.

8. The method according to claim 1, further comprising the steps of:
providing a second peptide digest from said subject comprising at least one M-protein peptide; wherein the quantity of said at least one M-protein peptide in said second peptide digest is different or the same as the quantity in said peptide digest; wherein said second peptide digest is obtained by performing the steps of:
subjecting a second body sample of said subject to protein electrophoresis in a gel to separate proteins comprised in said body sample into different protein fractions, optionally followed by immunofixation electrophoresis (IFE) and further optionally involving immunostaining of the gel;
excising from said gel a gel part comprising, or suspected of comprising, M-protein from said second body sample;
performing an enzymatic digestion of proteins present in said gel part in order to provide a second peptide digest comprising at least one M-protein peptide;
labeling said peptide digest with a first tandem mass tag (TMT) label and labelling said second peptide digest with a second TMT label; wherein said first TMT label and second TMT label are of different mass;
mixing said labelled first and labelled second peptide digest; and
subjecting said mixed labelled first and labelled second peptide digest, both comprising said at least one M-protein peptide, to liquid chromatography-mass spectrometry (LC-MS)
quantifying said M-protein in said second body sample as a relative quantity of said M-protein in said first body sample, whereby the M-protein in the first and second sample are quantified by their respective M-protein peptides which are of different mass and have the same LC retention time.

9. The method according to claim 8, wherein said first body sample of said subject is obtained while said subject is in remission, and wherein said second body sample of said subject is obtained while said subject is in relapse.

10. The method according to claim 1, wherein said plasma cell dyscrasia is selected from the group consisting of leukemia or lymphoma of B-cell non-Hodgkin type with plasma cell component; multiple myeloma (MM); plasmacytoma; lymphoplasmacytic lymphoma; AL amyloidosis; monoclonal gammopathy of undetermined significance (MGUS); smoldering multiple myeloma (SMM); macroglobulinemia; Waldenström disease; plasmacytoma; acute lymphoblastic leukemia (ALL); chronic lymphocytic leukemia (CLL); prolymphocytic leukemia (PLL); T-lymphoblastic lymphoma (TLL); acute myeloblastic leukaemia (AML); B-cell lymphoma, and cryoglobulinemia.

11. The method according to claim 1, wherein the subject has been treated with, or is undergoing treatment with, a therapeutic monoclonal antibody or small molecule inhibitor, wherein said therapeutic monoclonal antibody is daratumumab, nivolumab, elotuzumab, denosumab, blinatumomab or ipilimumab.

12. The method according to claim 1, wherein said MS is high-resolution MS.

13. The method according to claim 1, wherein said enzymatic digestion is performed by using a protease.

14. A method for typing a subject in plasma cell dyscrasia remission as having plasma cell dyscrasia minimal residual disease (MRD), comprising performing the method of claim 1 on a body sample of said subject, wherein said subject is typed as having plasma cell dyscrasia MRD if M-protein is detected in said body sample.

15. A method for monitoring a subject having plasma cell dyscrasia MRD, said method comprising performing a method of quantifying according to claim 1 on at least two longitudinal body samples that have been retrieved at different time points that are spaced apart by an interval of at least one or more days.

16. (canceled)

17. A method of treating a subject suffering from plasma cell dyscrasia or having plasma cell dyscrasia MRD, comprising the step of:
performing the method of claim 1 in order to determine that M-proteins are present in said subject, and
administering to said subject a therapeutically effective amount of a standard-of-care therapeutic agent against plasma cell dyscrasia comprising a therapeutic monoclonal antibody or small molecule inhibitor.

18. (canceled)

19. (canceled)

20. The method of claim 1 wherein the protein electrophoresis in a gel is serum protein electrophoresis (SPE) in an agarose gel.

21. The method of claim 2 wherein the protein electrophoresis in a gel is serum protein electrophoresis (SPE) in an agarose gel.
